(19) United States
(12) Patent Application Publication
(10) Pub. No.: US 2023/0125232 A1
(43) Pub. Date: Apr. 27, 2023

(54) ATP-DEPENDENT DNA LIGASE

(71) Applicant: ARCTICZYMES AS, Tromsø (NO)

(72) Inventors: Bernd Ketelsen Striberny, Kvaløya (NO); Terese Solstad, Kvaløysletta (NO); Olav Lanes, Tromsø (NO)

(21) Appl. No.: 17/912,770

(22) PCT Filed: Mar. 31, 2021
(86) PCT No.: PCT/EP2021/058452

§ 371 Date: Sep. 19, 2022

(30) Foreign Application Priority Data
Mar. 31, 2020 (EP) 20167251.6

(51) Int. Cl. C12N 9/00; C12N 15/63

(52) U.S. Cl. CPC C12N 9/93; C12Y 605/01001; C12N 15/63

(57) ABSTRACT

The present invention relates to the field of ligases. More specifically it relates to novel and highly efficient ATP-dependent DNA ligases with a unique ligase activity making the ligase particularly useful in a variety of molecular biology techniques. Furthermore, the invention relates to compositions and kits comprising the DNA ligase, methods for its manufacture and use.

### THE FIELD OF THE INVENTION

The present invention relates to the field of ligases. More specifically it relates to ATP-dependent DNA ligase, kits and compositions comprising the DNA ligase, method of manufacture and use.

### BACKGROUND

DNA ligases catalyzes the formation of phosphodiester bonds in the sugar-phosphate backbone of DNA between 5′-phosphoryl and 3′-hydroxyl end-groups in properly aligned DNA sequences. In vivo ligases play an important role in the repair of nicks as well as single-stranded and double-stranded breaks critical for DNA replication and repair.

In addition to be important for DNA replication and repair, isolated DNA ligases from bacteriophages, such as T4 DNA ligase, have for several decades been widely used in molecular biology for insertion of DNA fragments into vectors for recombinant plasmid constructions, adaptor ligation in next generation DNA sequencing library constructions and in circularization of dsDNA, cf. “Ligases”, Enzyme Resources Guide. Promega Corporation. pp. 8-14.

DNA ligase enzyme catalyze the covalent linkage of a 3′-hydroxyl end-group of one DNA chain with an adjacent 5′-phosphoryl end-group of another, coupled with the pyrophosphate hydrolysis of the cofactor ATP or NAD, Gumport, R. I., et al., Proc. Natl. Acad. Sci. USA (1971), vol. 68:2559-63.

Based on substrate preferences ligases may be classified as DNA ligases or RNA ligases, i.e. enzymes that creates phosphodiester bonds of either DNA or RNA, respectively, Tomkinson, A. E. et al, Location of the active site for enzyme-adenylate formation in DNA ligase, Proc. Natl. Acad. Sci. USA, 1991, vol. 88, p. 400-404.

Sealing single-stranded break in a double stranded nucleic acid molecule such as ligating prehybridized sticky-ends in a dsDNA molecule generated by asymmetric restriction enzyme digestions, is a lot more efficient than blunt end ligation where the two ends to be ligated are free in solution. A ligation reaction catalyzed by a template-dependent ligase, that catalyzes ligation of two single stranded nucleic acid molecules in the presence of a complementary nucleic acid molecule that spans the ligation junction and thus bringing the two ends in close proximity is a lot faster and more efficient than blunt end ligation.

Several DNA ligases and RNA ligases isolated from bacteriophages T3, T4 and T7 have been isolated and characterized within the last decades, Dunn, J. J., et al., J. Mal. Biol., 148:303-30 (1981); Armstrong, J., et al., Nucleic Acids Res., 11:7145-56 (1983); and Schmitt, M. P., et al., J. Mal. Biol., vol. 193:479-95 (1987). In vitro experiments using plasmids or oligonucleotides have revealed that T4 nucleic acid ligases join nicks in double-stranded nucleic acids with varying efficiency and substrate specificity, cf. Bullard, D. R., & Bowater, R. P, Direct comparison of nick-joining activity of the nucleic acid ligases from bacteriophage T4, The Biochemical Journal, 2006, 398(1), 135-144. The study demonstrated that T4 RNA (T4RnL) ligases and T4 DNA(T4DnL) ligases are able to ligate a variety of DNA-RNA hybrids. A DNA ligase isolated from Vaccinia virus or from eucaryotic L cell has been reported to seal nicks in DNA-RNA hybrids, cf. Sekiguchi, J. and Shuman, S., Ligation of RNA-containing duplexes by Vaccinia DNA ligase, Biochemistry, 1997, vol. 36, 9073-9079 and Bedows, E. et al., L cell DNA ligase joins RNA to DNA on a DNA template, Biochemistry, 1997, vol. 16, no. 10, 2231-2235.

However, ligation with high efficiency a DNA molecule to the 5′end of a RNA molecule in the presence of a complementary DNA molecule spanning the ligation junction have so far not been described in literature, cf. for example Bullard and Bowater, 2006, Sekiguchi and Shuman, 1997 and Bedows et al., 1977.

Despite the existence of several nucleic acid ligases suitable for use in recombinant DNA technology, there is an ever-existing need for additional highly efficient ligases with unique substrate specific ligation properties.

The present inventors have solved the above-mentioned need by cloning and recombinant expression and isolation of a new family of ATP-dependent DNA ligases isolated from bacteriophage which have been shown by biochemical characterization to be able to ligate with high efficiency a single stranded DNA molecule to the 5′end of a RNA molecule in the presence of a complementary single stranded DNA template that spans the ligation junction.

The ligases according to the present invention have in addition to the above described unique substrate specificity also the ability to ligate with high efficiency a single stranded DNA molecule to the 3′end of a RNA molecule in the presence of a complementary single stranded DNA template that spans the ligation junction.

### SUMMARY OF THE INVENTION

According to a first aspect the present invention provides an isolated ATP-dependent DNA ligase or an enzymatically active fragment thereof, wherein the DNA ligase comprises the amino acid sequence of SEQ ID No. 1 or comprises an amino acid sequence having at least 70% amino acid sequence identity to SEQ ID No. 1 and wherein the DNA ligase is able to ligate a 3′-hydroxyl-deoxyribonucleic acid molecule to a 5′-end of a 5′phosphoryl-ribonucleic acid molecule in the presence of a complementary deoxyribonucleic acid molecule that spans the ligation junction.

In one embodiment of the first aspect the isolated ATP-dependent DNA ligase or an enzymatically active fragment thereof comprises an amino acid sequence which is at least 75% identical to SEQ ID No. 1.

In one embodiment of the first aspect the isolated ATP-dependent DNA ligase or an enzymatically active fragment thereof comprises an amino acid sequence having at least 80%, 85%, 90%, 93% or 95% amino acid sequence identity to SEQ ID No. 1. In one further embodiment of the first aspect the isolated ATP-dependent DNA ligase consists of amino acid sequence with SEQ ID No. 1. Enzymatically active fragment thereof are also provided wherein the DNA ligase fragments are able to ligate a 3′-hydroxyl-deoxyribonucleic acid molecule to a 5′-end of a 5′phosphoryl-ribonucleic acid molecule in the presence of a complementary deoxyribonucleic acid molecule that spans the ligation junction.

In one further embodiment of the first aspect an isolated ATP-dependent DNA ligase with an amino acid sequence which is at least 70% identical to SEQ ID No. 1 is a DNA ligase isolated from a phage. Preferably from Cronobacter phage, Pectobacterium phage or Acinetobacter phage.

In another embodiment according to the present invention the ATP dependent DNA ligase that comprises an amino acid sequence which is at least 70% identical to SEQ ID No. 1 (L13) is selected from any of the DNA ligases with SEQ ID No. 7, SEQ ID No. 10 and SEQ ID No. 16.

In one further aspect there is provided an ATP-dependent DNA ligase or an enzymatically active fragment thereof, wherein said DNA ligase has an amino acid sequence selected from:

(a) SEQ ID No. 1 or an amino acid sequence which is at least 80% identical thereto,

(b) SEQ ID No. 7 or an amino acid sequence which is at least 80% identical thereto,

(c) SEQ ID No. 10 or an amino acid sequence which is at least 80% identical thereto, or

(d) SEQ ID No. 16 or an amino acid sequence which is at least 80% identical thereto and

wherein the DNA ligase is able to ligate a 3′-hydroxyl-deoxyribonucleic acid molecule to a 5′-end of a 5′phosphoryl-ribonucleic acid molecule in the presence of a complementary deoxyribonucleic acid molecule that spans the ligation junction.

In preferred embodiments of this aspect of the invention, the ATP-dependent DNA ligase has an amino acid sequence which is at least 85%, 90% or 95%, e.g. at least 98% or 99% or 99.5%, identical to SEQ ID NOs. 1, 7, 10 or 16.

In one embodiment of the first aspect the isolated ATP-dependent DNA ligase or an enzymatically active fragment thereof, wherein said DNA ligase has an amino acid sequence selected from any one of SEQ ID No. 1, SEQ ID No. 7, SEQ ID No. 10 and SEQ ID No. 16.

In one embodiment of the first aspect the isolated ATP-dependent DNA ligase or an enzymatically active fragment thereof comprising a His-tag, wherein said DNA ligase has an amino acid sequence selected from any one of SEQ ID No. 2, SEQ ID No. 8, SEQ ID No. 11 and SEQ ID No. 17.

In one embodiment of the first aspect the isolated ATP-dependent DNA ligase has the amino acid sequence of SEQ ID No. 1 or SEQ ID No. 2.

In one embodiment of the first aspect the isolated ATP-dependent DNA ligase has the amino acid sequence of SEQ ID No. 7 or SEQ ID No. 8.

In one embodiment of the first aspect the isolated ATP-dependent DNA ligase has the amino acid sequence of SEQ ID No. 10 or SEQ ID No. 11.

In one embodiment of the first aspect the isolated ATP-dependent DNA ligase has the amino acid sequence of SEQ ID No. 16 or SEQ ID No. 17.

According to one embodiment of the first aspect the ATP-dependent DNA ligase is a ligase providing a conversion rate providing a ligated product on a substrate comprising a single-stranded break in a double-stranded nucleic acid molecule wherein the double-stranded nucleic acid molecule comprises a 3′-hydroxyl-deoxyribonucleic acid molecule and the 5′-phosphoryl ribonucleic acid in complex with a complementary deoxyribonucleic acid molecule that spans the ligation junction wherein the conversion rate is at least 0.02, at least 0.03, at least 0.04, at least 0.05 or at least 0.1 wherein the conversion rate is determined as pmol substrate per pmol enzyme per minute at 25° C. to 30° C. in a ligation buffer comprising ATP, Mg²⁺ and from 0.2 pmol to 15 pmol of a DNA ligase according to the present invention.

According to one embodiment of the first aspect the ATP-dependent DNA ligase is a ligase providing a conversion rate providing a ligated product on a substrate comprising a single-stranded break in a double-stranded nucleic acid molecule wherein the double-stranded nucleic acid molecule comprises a 3′-hydroxyl-deoxyribonucleic acid molecule and the 5′-phosphoryl ribonucleic acid in complex with a complementary deoxyribonucleic acid molecule that spans the ligation junction wherein the conversion is at least 0.15, at least 0.2, at least 0.3, at least 0.4, at least 0.5, at least 0.8, at least 1.0, at least 1.5 wherein the conversion rate is determined as pmol substrate per pmol enzyme per minute at 25° C. to 30° C. in a ligation buffer comprising ATP, Mn²⁺ and 0.2 pmol to 15 pmol of a DNA ligase according to the present invention.

In one embodiment of the first aspect the ATP-dependent DNA ligase comprises a tetrapeptide motif KXaaDG wherein Xaa is an aliphatic and nonpolar amino acid and wherein the tetrapeptide motif corresponds to amino acid position 159 to 162, the numbering being in accordance with the amino acid numbering in SEQ ID No. 1.

In one further embodiment the aliphatic and nonpolar amino acid residue is selected from the group consisting of Methionine, Isoleucine, Leucine, Valine, Leucine, Alanine and Glycine.

In yet a further embodiment the aliphatic and nonpolar amino acid residue is Methionine.

In another embodiment of the first aspect the isolated ATP-dependent DNA ligase or an enzymatically active fragment thereof is derived from a bacteriophage.

In a second aspect of the present invention there is provided a recombinant nucleic acid molecule encoding the isolated ATP-dependent DNA ligase or an enzymatically fragment thereof according to the first aspect or encoding a protein comprising said isolated ATP-dependent DNA ligase or an enzymatically fragment thereof.

In on embodiment of the second aspect the recombinant nucleic acid molecule comprises or consists of a nucleic acid molecule of SEQ ID No. 3 or a codon-optimized nucleic acid sequence of SEQ ID No. 3.

In on embodiment of the second aspect the recombinant nucleic acid molecule comprises or consists of a nucleic acid molecule of SEQ ID No. 9 or a codon-optimized nuclei acid sequence of SEQ ID No. 9.

In on embodiment of the second aspect the recombinant nucleic acid molecule comprises or consists of a nucleic acid molecule of SEQ ID No. 18 or a codon-optimized nuclei acid sequence of SEQ ID No. 18.

In on embodiment of the second aspect the recombinant nucleic acid molecule comprises or consists of a nucleic acid molecule selected from any one of SEQ ID No. 3, SEQ ID No. 9, SEQ ID No. 12 and SEQ ID No. 18 or a codon-optimized nuclei acid sequence of SEQ ID No. 3, SEQ ID No. 9, SEQ ID No. 12 and SEQ ID No. 18.

In on embodiment of the second aspect the recombinant nucleic acid molecule comprises or consists of a nucleic acid molecule selected from any one of SEQ ID No. 3, SEQ ID No. 9, SEQ ID No. 12 and SEQ ID No. 18 or a degenerated version of SEQ ID No. 3, SEQ ID No. 9, SEQ ID No. 12 and SEQ ID No. 18.

In a third aspect of the present invention there is provided a vector comprising a nucleic acid molecule encoding the isolated ATP-dependent DNA ligase or an enzymatically active fragment thereof according to the first aspect or comprising the recombinant nucleic acid molecule according to the second aspect, wherein the vector is a recombinant expression vector, a cloning vector, a plasmid, a viral vector, a cosmid, a lambda phage or a bacterial artificial chromosome.

In one embodiment of the third aspect there is provided a vector comprising or consisting a nucleic acid molecule
a) encoding an ATP-dependent DNA ligase or an enzymatically active fragment thereof wherein the ATP-dependent DNA ligase has an amino acid sequence of SEQ ID No. 1 or an amino acid sequence having at least 70% amino acid sequence identity to SEQ ID No. 1 or
b) comprising a nucleic acid molecule of SEQ ID No. 3 or a codon-optimized nucleic acid sequence of SEQ ID No. 3 encoding the ATP-dependent DNA ligase in a) and
c) wherein the ATP-dependent DNA ligase in a) orb) is able to ligate a 3′-hydroxyl-deoxyribonucleic acid molecule to a 5′-end of a 5′phosphoryl-ribonucleic acid molecule in the presence of a complementary deoxyribonucleic acid molecule that spans the ligation junction.

In one embodiment of the third aspect there is provided a vector comprising or consisting a nucleic acid molecule
a) encoding an ATP-dependent DNA ligase or an enzymatically active fragment thereof wherein the ATP-dependent DNA ligase has an amino acid sequence of SEQ ID No. 1 or an amino acid sequence having at least 75% amino acid sequence identity to SEQ ID No. 1 or
b) comprising a nucleic acid molecule of SEQ ID No. 3 or a codon-optimized nucleic acid sequence of SEQ ID No. 3 encoding the ATP-dependent DNA ligase in a) and
c) wherein the ATP-dependent DNA ligase in a) orb) is able to ligate a 3′-hydroxyl-deoxyribonucleic acid molecule to a 5′-end of a 5′phosphoryl-ribonucleic acid molecule in the presence of a complementary deoxyribonucleic acid molecule that spans the ligation junction.

In one embodiment of the third aspect there is provided a vector comprising or consisting a nucleic acid molecule
a) encoding an ATP-dependent DNA ligase or an enzymatically active fragment thereof wherein the ATP-dependent DNA ligase has an amino acid sequence selected from any one of SEQ ID No. 1, SEQ ID No. 7, SEQ ID No. 10 and SEQ ID No. 16 or
b) encoding an ATP-dependent DNA ligase or an enzymatically active fragment thereof wherein the ATP-dependent DNA ligase has an amino acid sequence selected from: SEQ ID No. 1 or an amino acid sequence which is at least 80% identical thereto, SEQ ID No. 7 or an amino acid sequence which is at least 80% identical thereto, SEQ ID No. 10 or an amino acid sequence which is at least 80% identical thereto, or SEQ ID No. 16 or an amino acid sequence which is at least 80% identical thereto or
c) comprising a nucleic acid molecule selected from any one of SEQ ID No. 3, SEQ ID No. 9, SEQ ID No. 12 and SEQ ID No. 18 or a codon-optimized nucleic acid sequence of SEQ ID No. 3, SEQ ID No. 9, SEQ ID No. 12 and SEQ ID No. 18 encoding the ATP-dependent DNA ligase in a) and
d) wherein the ATP-dependent DNA ligase in a) orb) is able to ligate a 3′-hydroxyl-deoxyribonucleic acid molecule to a 5′-end of a 5′phosphoryl-ribonucleic acid molecule in the presence of a complementary deoxyribonucleic acid molecule that spans the ligation junction.

In one embodiment of the third aspect the vector is a recombinant expression vector, a cloning vector, a plasmid, a viral vector, a cosmid, a lambda phage or a bacterial artificial chromosome.

In one embodiment of the third aspect the vector is a recombinant expression vector.

In one embodiment of the third aspect the vector is preferably a plasmid.

In a fourth aspect of the present invention there is provided a host cell comprising the vector according to the third aspect wherein the host cell, is a yeast cell, insect cell, a human cell line or a bacterial cell.

In one embodiment of the fourth aspect the bacterial cell is preferably E. coli.

In a fifth aspect of the present invention there is provided a method for isolation and purification of the ATP-dependent DNA ligase or the enzymatically active fragment thereof according to the first aspects and embodiments thereof, comprising the steps of:
a) culturing the host cell according to the fourth aspect, under conditions suitable for the expression of the ATP-dependent DNA ligase or the enzymatically active fragment thereof according to the first aspects and embodiments thereof; and
b) isolating the DNA ligase or the enzymatically active fragment thereof from the host cell or from the culture medium or supernatant.

In a sixth aspect of the present invention there is provided a composition comprising the ATP-dependent DNA ligase or an enzymatically fragment thereof according to the first aspect.

In one embodiment of the sixth aspect the composition further comprises a buffer.

The buffer may in one embodiment be a buffer comprising ATP and Mg²⁺ or Mn²⁺.

The buffer may in one embodiment be a buffer comprising ATP and MgCl₂ or MnCl₂.

The buffer may in one embodiment be a buffer suitable for storing the ATP-dependent DNA ligase according to the invention, wherein the buffer comprises Tris-HCl, KCl, Mg²⁺, BSA and glycerol.

In one embodiment of the sixth aspect the composition further comprises at least one first 3′-hydroxyl-deoxyribonucleic acid molecule, at least one 5′phosphoryl-ribonucleic molecule and at least one second complementary deoxyribonucleic acid molecule wherein the DNA ligase is able to ligate the at least one first 3′-hydroxyl-deoxyribonucleic acid molecule to the 5′end of the at least one 5′phosphoryl-ribonucleic molecule in the presence of the complementary deoxyribonucleic acid molecule that spans the ligation junction.

In one embodiment of the sixth aspect the at least one first 3′-hydroxyl-deoxyribonucleic acid molecule is immobilized on a bead or further comprises a capturing label preferably biotin or a derivatized nucleotide such as a dye, preferably a fluorescent dye wherein the bead, the capturing label or the derivatized nucleotide is attached to the 5′end of said first deoxyribonucleic acid molecule.

In one embodiment of the sixth aspect the composition further comprising a ligation buffer comprising ATP.

In one further embodiment the concentration of ATP of the ligation buffer is from about 0.01 mM to about 10 mM ATP and preferably from about 0.05 mM to about 2.5 mM ATP.

In one further embodiment the ligation buffer comprises a divalent cation.

In one further embodiment the divalent cation of the ligation buffer is Mg²⁺ or Mn²⁺ for example in form of MgCl₂ or MnCl₂ and wherein the concentration of Mg²⁺ or Mn²⁺ is from about 1 mM to about 20 mM, preferably from about 5 mM to about 10 mM.

In one further embodiment the divalent cation of the ligation buffer is Mg²⁺ for example in form of MgCl₂ and wherein the concentration of Mg²⁺ is from about 1 mM to about 20 mM, preferably from about 5 mM to about 10 mM.

In one further embodiment the divalent cation of the ligation buffer is Mn²⁺ for example in form of MnCl₂ and wherein the concentration of Mn²⁺ is from about 1 mM to about 20 mM, preferably from about 5 mM to about 10 mM.

In one further embodiment the ligation buffer comprises ATP in a concentration of from about 0.01 mM to about 10 mM ATP and preferably from about 0.05 mM to about 2.5 mM ATP and Mg²⁺ for example in form of MgCl₂ and wherein the concentration of Mg²⁺ is from about 1 mM to about 20 mM, preferably from about 5 mM to about 10 mM.

In one further embodiment the ligation buffer comprises ATP in a concentration of from about 0.01 mM to about 10 mM ATP and preferably from about 0.05 mM to about 2.5 mM ATP and Mn²⁺ for example in form of MnCl₂ and wherein the concentration of Mn²⁺ is from about 1 mM to about 20 mM, preferably from about 5 mM to about 10 mM.

In a seventh aspect of the present invention there is provided a kit for ligating a deoxyribonucleic acid molecule to a terminus of a ribonucleic acid molecule comprising

a. a first container comprising the isolated ATP-dependent DNA ligase or an enzymatically fragment thereof according to the first aspect or a composition comprising the ATP-dependent DNA ligase according to the sixth aspect;

b. a second container comprising a ligation buffer comprising ATP and a divalent cation;

c. optionally a third container comprising at least one first 3′-hydroxyl-deoxyribonucleic acid molecule to be ligated to the 5′end of a 5′phosphoryl-ribonucleic acid molecule, and at least one second deoxyribonucleic molecule wherein the at least one second deoxyribonucleic acid molecule comprises a 3′region and a 5′region wherein the 3′region is complementary to the first deoxyribonucleic acid molecule and the 5′ region is either a sequence that is complementary to a ribonucleic acid molecule comprising a known sequence or the 5′region is a sequence that is degenerated in order to bind ribonucleic acid molecules having an unknown sequence or comprising different sequences and wherein the first deoxyribonucleic molecule and the second deoxyribonucleic molecule is in the form of a prehybridized complex; and

d. optionally instructions for using the kit.

In one embodiment of the seventh aspect the kit comprises a fourth container comprising at least one first 5′-phosphoryl-deoxyribonucleic acid molecule to be ligated to the 3′end of a 3′-hydroxyl-ribonucleic acid molecule, and at least one second deoxyribonucleic molecule wherein the at least one second deoxyribonucleic acid molecule comprises a 3′region and a 5′region wherein the 5′region is complementary to the first deoxyribonucleic acid molecule and the 3′ region is either a sequence that is complementary to a ribonucleic acid molecule comprising a known sequence or the 3′region is a sequence that is degenerated in order to bind ribonucleic acid molecules having an unknown sequence or comprising different sequences and wherein the first 5′-phosphoryl-deoxyribonucleic acid and the second deoxyribonucleic molecule may be in the form of a prehybridized complex.

In one embodiment the concentration of ATP and the divalent cation of the ligation buffer is from about 0.01 mM to about 10 mM ATP and preferably from about 0.05 mM to about 2.5 mM ATP and the divalent cation is Mg²⁺ or Mn²⁺ for example in form of MgCl₂ or MnCl₂ and wherein the concentration of Mg²⁺ or Mn²⁺ is from about 1 mM to about 20 mM, preferably from about 5 mM to about 10 mM. Preferably the divalent cation is Mn²⁺ in form of MnCl₂.

In a further aspect of the present invention there is provided method for ligating a single-stranded break in a double-stranded nucleic acid molecule wherein said method comprising contacting the double-stranded nucleic acid molecule comprising a single stranded break with the isolated ATP-dependent DNA ligase or enzymatically active fragment thereof according to the first aspect or the composition according to the sixth aspect.

In an eighth aspect of the present invention there is provided a method for ligating a single-stranded break in a double-stranded nucleic acid molecule, wherein said method comprising contacting a double-stranded nucleic acid molecule comprising a single stranded break with the isolated ATP-dependent DNA ligase or enzymatically active fragment thereof according to the first aspects and embodiments thereof under conditions which permits ligation of the 3′-hydroxyl-deoxyribonucleic acid molecule to the 5′end of the 5′-phosphoryl ribonucleic acid molecule in the double-stranded nucleic acid molecule wherein 3′-hydroxyl-deoxyribonucleic acid molecule and the 5′-phosphoryl ribonucleic acid is in complex with a complementary deoxyribonucleic acid molecule that spans the ligation junction.

In one embodiment according to the above method, the 3′-hydroxyl-deoxyribonucleic acid molecule is immobilized on a bead or further comprises a capturing label preferably biotin or a derivatized nucleotide such as a dye, preferably a fluorescent dye wherein the bead, the capturing label or the derivatized nucleotide is linked to the 5′end of said 3′-hydroxyl-deoxyribonucleic acid molecule.

In one embodiment according to the above method the ribonucleic acid (RNA) molecule is a RNA molecule selected from a group comprising messenger RNA (mRNA), ribosomal RNA (rRNA), microRNA (miRNA), short interfering RNA (siRNA), repeat associated siRNA (rasiRNA).

In one embodiment according to the above method further comprises adding a ligation buffer comprising ATP and divalent cation.

In one further embodiment according to the above method the concentration of ATP is from about 0.01 mM to about 10 mM and preferably from about 0.05 mM to about 2.5 mM and wherein divalent cation is Mg²⁺ or Mn²⁺ for example in form of MgCl₂ or MnCl₂ and wherein the concentration of Mg²⁺ or Mn²⁺ is from about 1 mM to about 20 mM, preferably from about 5 mM to about 10 mM. Preferably the divalent cation is Mn²⁺ in form of MnCl₂.

In one embodiment according to the above method further comprises incubating the ATP dependent DNA ligase together with the double stranded nucleic acid molecule comprising a single stranded break within less than 6 hours, such as within less 1 hour, such as with in less than 45 minutes or preferably within 30 minutes, more preferably within 15 minutes to achieve at least 50%, at least 55%, at least 60%, at least 65% at least 70%, at least 75%, at least 80%, at least 85%, at least 90%, at least 95% or at least 100% ligation of the nucleic acid fragments.

In one embodiment according to the above method wherein the method is used for RNA 5′-end adapter ligation, for capturing RNA molecules of known or unknown sequences, ligation of an DNA element that serves as a template in cDNA molecule synthesis comprising promoter elements and/or translation enhancer elements to 5′ends of RNA molecules for the purpose of in vitro transcription.

The ninth aspect of the present invention provides use of the ATP-dependent DNA ligase or an enzymatically active fragment thereof according to the first aspect or the composition comprising the ATP-dependent DNA ligase according to the sixth aspect for RNA 5′-end adapter ligation, for capturing RNA molecules of known or unknown sequences, ligation of an DNA element that serves as a template in cDNA molecule synthesis comprising promoter elements and/or translation enhancer elements to 5′ends of RNA molecules for the purpose of in vitro transcription.

In a tenth aspect of the present invention there is provided a method for ligating a deoxyribonucleic acid molecule to a 5′end and a 3′ end of ribonucleic acid molecules, the method comprising:

a. providing a sample comprising a population of ribonucleic acid molecules wherein one or more of the ribonucleic acid molecules comprises a 5′phosphoryl-end group and a 3′-hydroxyl-end group;

b. ligating at least one first 3′-hydroxyl-deoxyribonucleic acid molecule to the 5′end of the ribonucleic acid molecules in the presences of at least one second deoxyribonucleic wherein the at least one second deoxyribonucleic acid molecule comprises a 3′region and a 5′ region wherein the 3′region is complementary to the first deoxyribonucleic acid molecule and the 5′ region is either a sequence that is complementary to a ribonucleic acid molecule comprising a known sequence or the 5′region is a sequence that is degenerated in order to bind ribonucleic acid molecules having an unknown sequence or comprising different sequences and wherein the first deoxyribonucleic molecule and the second deoxyribonucleic molecule may be in the form of a prehybridized complex; and

c. ligating at least one other 5′phosphoryl-deoxyribonucleic acid molecule to the 3′-end of the ribonucleic acid molecules in step b. in the presences of at least one additional second deoxyribonucleic comprises a 3′region and a 5′ region wherein the 5′region is complementary to the 5′phosphoryl-deoxyribonucleic acid molecule and the 3′ region is either a sequence that is complementary to the ribonucleic acid molecule comprising a known sequence in step b. or the 3′region is a sequence that is degenerated in order to bind the ribonucleic acid molecules having an unknown sequence or comprising different sequences and wherein the 5′phosphoryl-deoxyribonucleic acid and the additional second deoxyribonucleic molecule may be in the form of a prehybridized complex; and

wherein the ligation reactions in step b and c are catalyzed by the ATP-dependent ligase according to the first aspect and wherein ligation reactions in step b and c are performed simultaneously or sequentially.

In one embodiment of the tenth aspect the sample further comprises ATP and a divalent cation, preferably Mn²⁺ or Mg²⁺.

A further aspect of the present invention provides use of the method of the tenth aspect in RNA library construction.

### DESCRIPTION OF THE FIGURES

FIG. 1: Sequence alignment comparing amino acid sequence of T4 DNA ligase and L13 ligase of the present invention.

FIG. 2: Multiple sequence alignment of the DNA ligases according to the present invention derived from different bacteriophages. All the sequences comprise the KXDG adenylation site, a common motif for all ligases identified so far.

FIG. 3a: Illustrates the general experimental set-up for measuring ligase activity which is based on the experimental set-up described in Bullard and Bowater, 2006.

FIG. 3b: Substrate specificity of AZ L13 compared to other known ligases. A set of eight different nicked substrates were used in order to test the enzyme activity of the ligase AZ L13 and the commercial reference enzymes (AZ T4 DNA Ligase (ArcticZymes), T4 Rnl 1 (NEB), T4 Rnl 2 (NEB), T3 DNA ligase (NEB), T7 DNA Ligase (NEB). The substrates used are each an assembly of three oligos that can either be composed of deoxyribonucleic acid (DNA) oligoes only (substrate S1) ribonucleic acids (RNA) oligoes only (substrate S2) or a mixture of deoxyribonucleic acid (DNA) oligoes and ribonucleic acids (RNA) oligoes (substrates S3 to S8). A positive ligase activity leads to an increase in the length of the fluorescent labelled nucleic acid oligo as depicted in FIG. 1b. The bands are represented by the lower bands being unligated single-stranded 8-mer oligonucleotide products and the upper bands being ligated single-stranded 20-mer oligonucleotide products.

FIG. 4: Depicts an aspect of the invention wherein DNA oligos are ligated using the ATP-dependent DNA L13 ligase enzyme of the present invention to 5′-ends of RNAs in the presence of DNA ligation templates wherein the DNA template sequence is semi-degenerated thus comprising a mixture of sequences. Wherein one region of the semi-degenerated DNA template is complementary to the first DNA oligo and the other region comprises one or more degenerated nucleotides for unspecific hybridization to different 5′-monophosphorylated RNA oligoes in order to capture RNA molecules of unknown sequences or in order to capture RNA molecules from different alleles wherein some nucleotide positions in the RNA oligos are different in different alleles. Alleles are gene variants representing the same gene. N denotes either A, C, G or T.

FIG. 5: Depicts an embodiment of the aspect described in FIG. 4 wherein the pool of RNA molecules comprises a mixture of monophosphorylated RNA molecules and RNA molecules comprising a 5′-modification, for example a 5′cap or a triple phosphate group. The RNA molecules comprising 5′ modification will be excluded from ligation.

FIG. 6: Depicts an alternative embodiment of the aspect of the invention described in FIG. 4 wherein the DNA molecule to be ligated to the monophosphorylated RNA molecules comprises a 5′ tag, illustrated in this figure as a Biotin tag. Capturing may be performed by using Biotin tags and Streptavidin coupled to a column or a magnetic bead for example.

FIG. 7: Depicts an alternative embodiment of the aspect of the invention described in FIG. 4 wherein the method capture specific mRNA species. As described above, there is a selective ligation of DNA oligos only to 5′-monophosphorylated ends of mRNA. mRNA is usually capped and not phosphorylated which would prevent ligation using the described L13 ligase. Dephosphorylation of all 5′-phosphate containing RNA species by phosphatases and subsequent de-capping of mRNA would prepare mRNA 5′-ends for ligation to 3′-ends of DNA. Capturing may be performed by using Biotin tags and Streptavidin.

FIG. 8: illustrates an alternative embodiment of an aspect of the invention wherein a promoter is tagged to de-capped mRNA for use in direct cell free ivTT (in vitro Transcription-Translation). Selective ligation of DNA oligos at the 5′-monophosphorylated ends of mRNA using a DNA template with degenerated region for unspecific hybridization as described in FIG. 4. Dephosphorylation of non-mRNA RNA species and de-capping of mRNA. 5′-phosphorylated mRNA will be tagged with a 5′-DNA adapter containing a degenerated region and a promoter sequence. First strand cDNA synthesis will lead to a RNA Polymerase-readable copy of the mRNA including a RNA Polymerase binding site so that the cDNA can be used directly in in vitro transcription/translation technologies.

FIG. 9a: Wild type amino acid sequence of the ATP-dependent DNA ligase according to the present invention (AZ L13) SEQ ID No 1.

FIG. 9b: His-tagged amino acid sequence of the ATP-dependent DNA ligase according to the present invention (AZ L13) SEQ ID No 2.

FIG. 9c: wildtype nucleotide sequence (SEQ ID No 3) encoding the amino sequence acid in FIG. 9a.

FIG. 10a: Amino acid sequence of the ATP-dependent DNA ligase according to the present invention (L13rel1) SEQ ID No 7.

FIG. 10b: His-tagged amino acid sequence of the ATP-dependent DNA ligase according to the present invention (L13rel1) SEQ ID No 8.

FIG. 10c: Wildtype nucleotide sequence (SEQ ID No 9) encoding the amino sequence acid in FIG. 10a.

FIG. 11a: Amino acid sequence of the ATP-dependent DNA ligase according to the present invention (L13rel2) SEQ ID No 10.

FIG. 11b: His-tagged amino acid sequence of the ATP-dependent DNA ligase according to the present invention (L13rel2) SEQ ID No 11.

FIG. 11c: Wildtype nucleotide sequence (SEQ ID No 12) encoding the amino sequence acid in FIG. 11a.

FIG. 12a: Amino acid sequence of an ATP-dependent DNA ligase (L13rel3) SEQ ID No 13.

FIG. 12b: His-tagged amino acid sequence of the ATP-dependent DNA ligase of FIG. 12a (L13rel3) SEQ ID No 14.

FIG. 12c: Wildtype nucleotide sequence (SEQ ID No 15) encoding the amino sequence acid in FIG. 12a.

FIG. 13a: Amino acid sequence of the ATP-dependent DNA ligase according to the present invention (L13rel4) SEQ ID No 16.

FIG. 13b: His-tagged amino acid sequence of the ATP-dependent DNA ligase according to the present invention (L13rel4) SEQ ID No 17.

FIG. 13b: Wildtype nucleotide sequence (SEQ ID No 18) encoding the amino sequence acid in FIG. 13a.

FIG. 14: Sequence alignment comparing amino acid sequence of Vaccinia DNA ligase and L13 ligase of the present invention.

### DETAILED DESCRIPTION OF THE INVENTION

In the following description, various examples and embodiments of the invention are set forth in order to provide the skilled person with a more thorough understanding of the invention. The specific details described in the context of the various embodiments and with reference to the attached drawings are not intended to be construed as limitations.

Unless specifically defined otherwise, all technical and scientific terms used herein have the same meaning as commonly understood by a skilled person in the fields of genetics, biochemistry, and molecular biology.

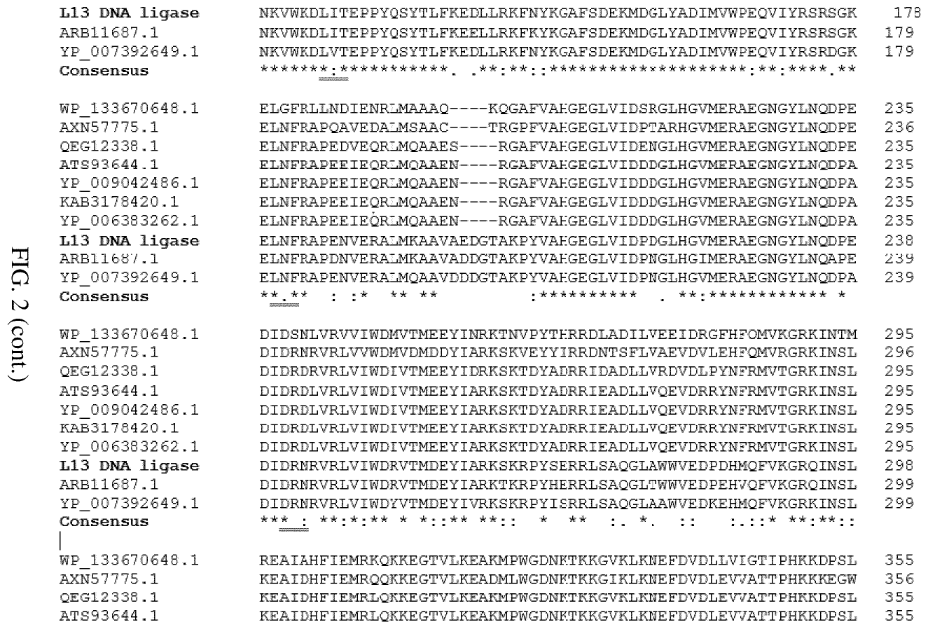

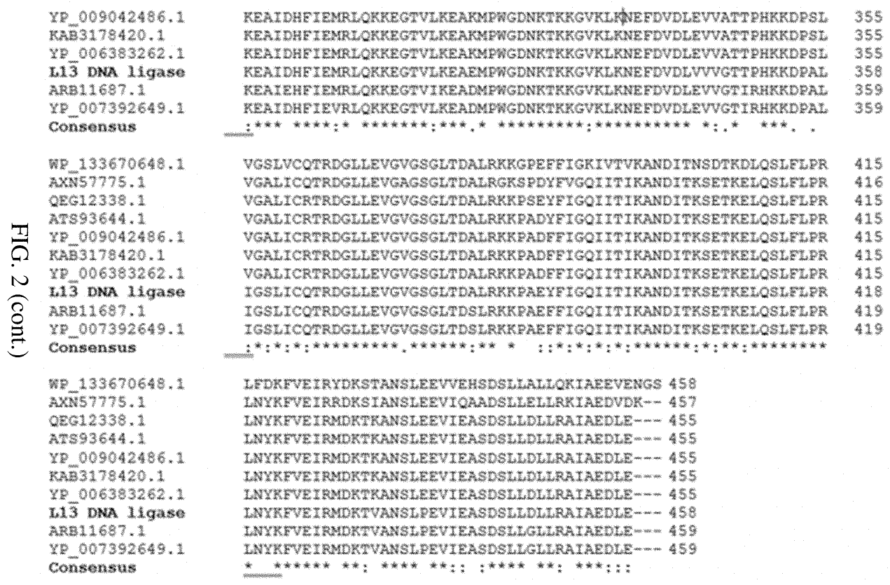

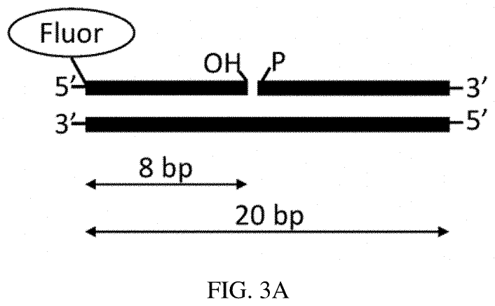

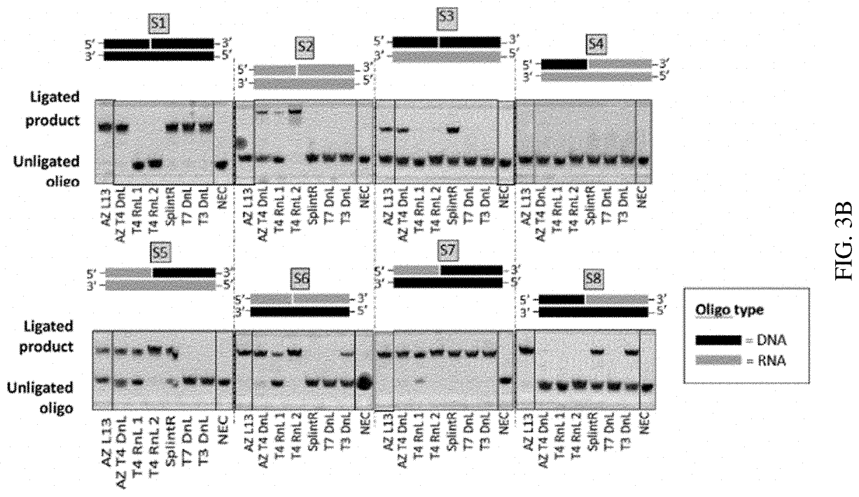

As mentioned above, the present inventors have identified a novel ATP-dependent DNA ligase (L13) with the ability to ligate a single stranded break in a double stranded nucleic acid complex comprising a first 3′-hydroxyl-deoxyribonucleic acid molecule to be ligated to a 5′end of a 5′phosphoryl-ribonucleic acid molecule in the presence of a complementary second deoxyribonucleic acid molecule that spans the ligation junction, for illustration see FIG. 3a.

The present inventors have also identified further DNA ligases originally isolated from bacteriophage wherein the further DNA ligases have an amino acid sequence which is at least 70% such at least 75% identical to the amino acid sequence of the L13 ligase with SEQ ID No. 1, wherein the further DNA ligases have similar enzyme activity and substrate specificity as L13.

The further DNA ligase is L13rel1 with amino acid sequence of SEQ ID No. 7 and cDNA sequence of SEQ ID No. 9.

The further DNA ligase is L13rel2 with amino acid sequence of SEQ ID No. 10 SEQ ID No. 12.

The further DNA ligase is L13rel4 with amino acid sequence of SEQ ID No. 16 SEQ ID No. 18.

The ligases according to the present invention have in addition to the above described unique substrate specificity also the ability to ligate with high efficiency a single-stranded break in a double stranded nucleic acid complex comprising a first DNA molecule comprising a 5′phosphoryl end-group to be ligated to the 3′end of a RNA molecule comprising a 3′-hydroxyl end-group in the presence of a complementary second deoxyribonucleic acid molecule that spans the ligation junction.

The first 3′-hydroxyl-deoxyribonucleic acid molecule, the 5′phosphoryl-ribonucleic acid molecule or portions thereof used in a ligation reaction together with a second deoxyribonucleic are preferably single stranded and the 3′-hydroxyl-deoxyribonucleic acid molecule and the 5′phosphoryl-ribonucleic acid molecule may be partially or wholly complementary to at least a portion of the second deoxyribonucleic molecule that spans the ligation junction.

The fact that “complementary nucleic acid sequences bind to each other” is a property of DNA and RNA. Complementarity is achieved by distinct interactions between nucleobases: adenine (A), thymine (T) or (uracil in RNA), guanine (G) and cytosine (C).

The term “3′-hydroxyl-deoxyribonucleic acid molecule” has a free hydroxyl group (OH-group) at its 3′ end. The nucleotides of the deoxyribonucleic acid molecule may be standard as well as nonstandard nucleotides. Non-limiting examples of nonstandard nucleotides include inosine, xanthosine, iso-guanosine, iso-cytidine, diaminopyrimidine, and deoxy-uridine. The deoxyribonucleic acid molecule may comprise modified or derivatized nucleotides. Non-limiting examples of modifications on the deoxyribose or base moieties include the addition (or removal) of acetyl groups, amino groups, carboxyl groups, carboxymethyl groups, hydroxyl groups, methyl groups, phosphoryl groups, and thiol groups. Suitable examples of derivatized nucleotides include those with covalently attached dyes, such as fluorescent dyes or quenching dyes, or other molecules, such as biotin, digoxygenin, or a magnetic particle, for illustrations see FIGS. 6 and 7.

The 3′-hydroxyl-deoxyribonucleic acid molecule may be linked to a magnetic bead, a glass or silica substrate or a surface in a microfluidic device or other reaction chamber at its 5′end. Additional solid-state substrates to which 3′-hydroxyl-deoxyribonucleic acid molecule can be coupled, directly or indirectly include acrylamide, cellulose, nitrocellulose, glass or other well-known substrates known to a skilled person.

The length of the 3′-hydroxyl-deoxyribonucleic acid molecule can and will vary dependent upon the length of the ligation product and its desired features. In general, the 3′-hydroxyl-deoxyribonucleic acid molecule is at least about 8 nucleotides, as shown in example 2, but may range from 15 nucleotides and up to 100 nucleotides in length.

5′Adapter Ligation of RNA

The term “complementary second deoxyribonucleic acid molecule” or “complementary ligation template” is to be understood as a deoxyribonucleic acid (DNA) molecule that is used to improve efficiency of the ligation reaction. Ligation of the first 3′-hydroxyl-deoxyribonucleic acid molecule at a 5′-end of the 5′phosphoryl-ribonucleic acid molecule is performed, as described above, in the presence of this second deoxyribonucleic acid molecule also called ligation template. This second deoxyribonucleic acid molecule comprises two distinct regions: a 5′ region that is complementary to and hybridizes with the ribonucleic acid molecule and a 3′ region that is complementary to and hybridizes with the first deoxyribonucleic acid molecule, see FIG. 3a.

The “complementary second deoxyribonucleic acid molecule” may be an exact complement or it may be a nearly exact complement of its two target sequences. Since the ligation template hybridizes to both the first 3′-hydroxyl-deoxyribonucleic acid molecule and the ribonucleic acid molecule, it spans the ligation junction, such that the 3′-hydroxyl-deoxyribonucleic acid molecule is brought into close proximity to the 5′ end of the 5′phosphoryl-ribonucleic acid molecule. The second deoxyribonucleic acid molecule may also comprise standard, nonstandard, modified or derivatized nucleotides similar as described for the 3′-hydroxyl-deoxyribonucleic acid molecule.

In general, the complementary second deoxyribonucleic acid molecule will be at least about 10 nucleotides in length, preferably at least about 20 nucleotides in length, with about half of the ligation template having complementarity to the ribonucleic acid molecule and the other half having complementarity to the first 3′-hydroxyl-deoxyribonucleic molecule. A person skilled in the art will appreciate that the complementary second deoxyribonucleic acid molecule may be longer for example at least about 25 or 30 or 35 or 40 or 45 or up to about 100 nucleotides.

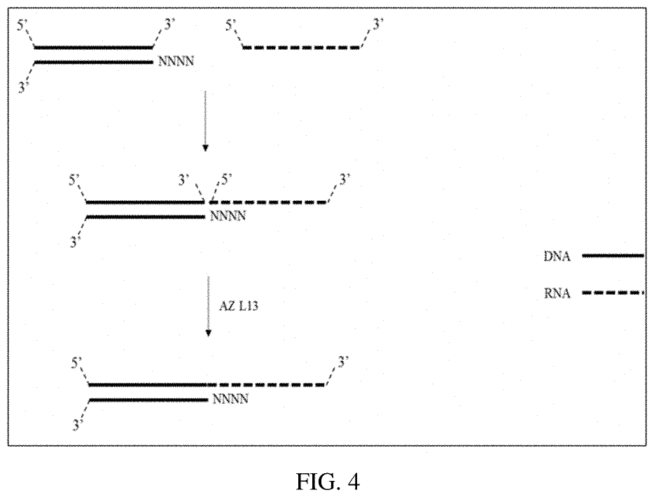

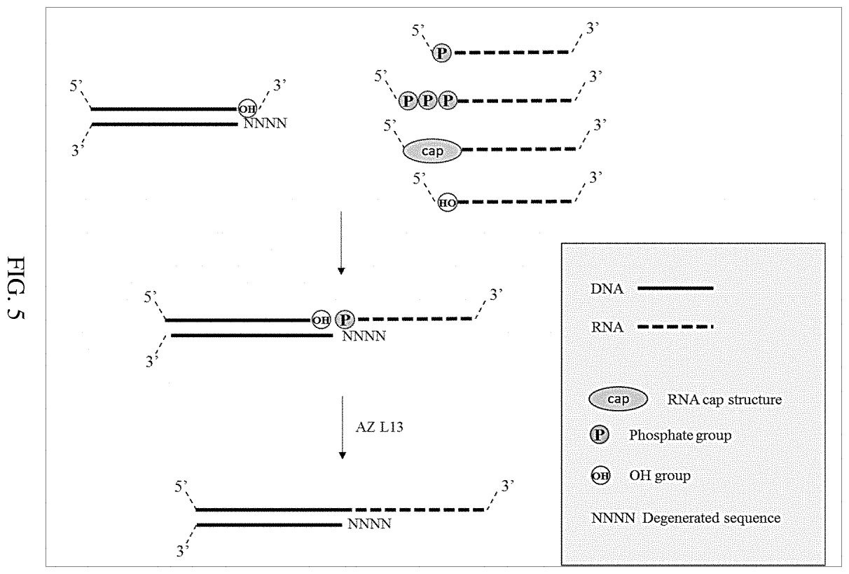

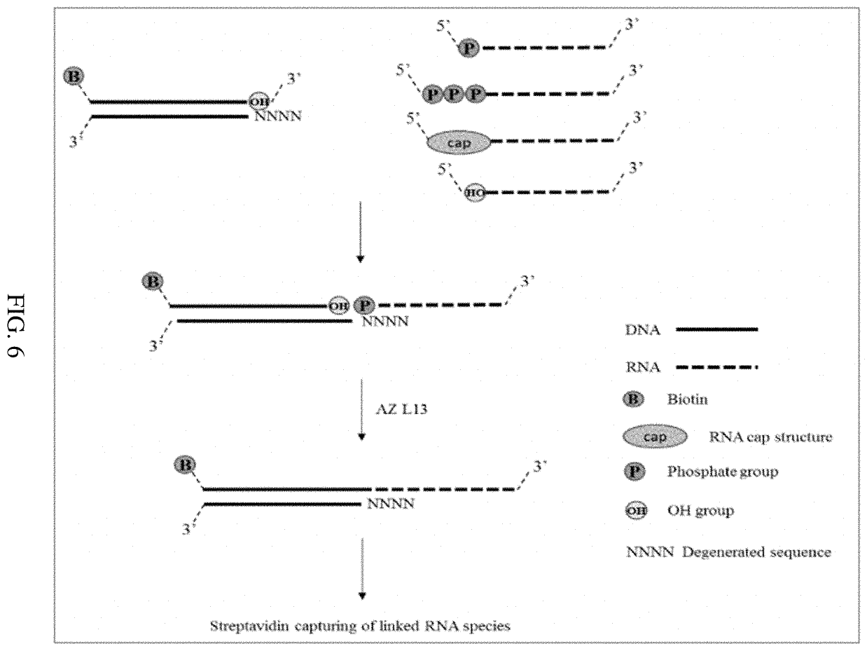

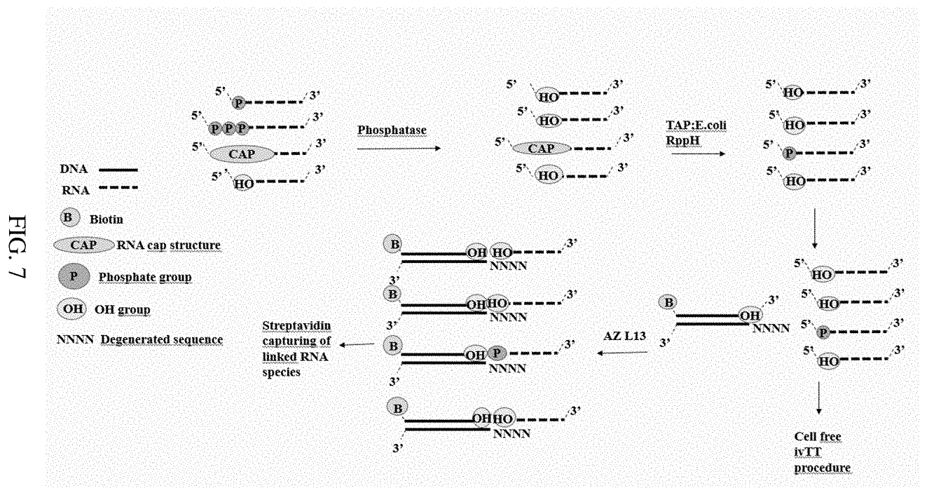

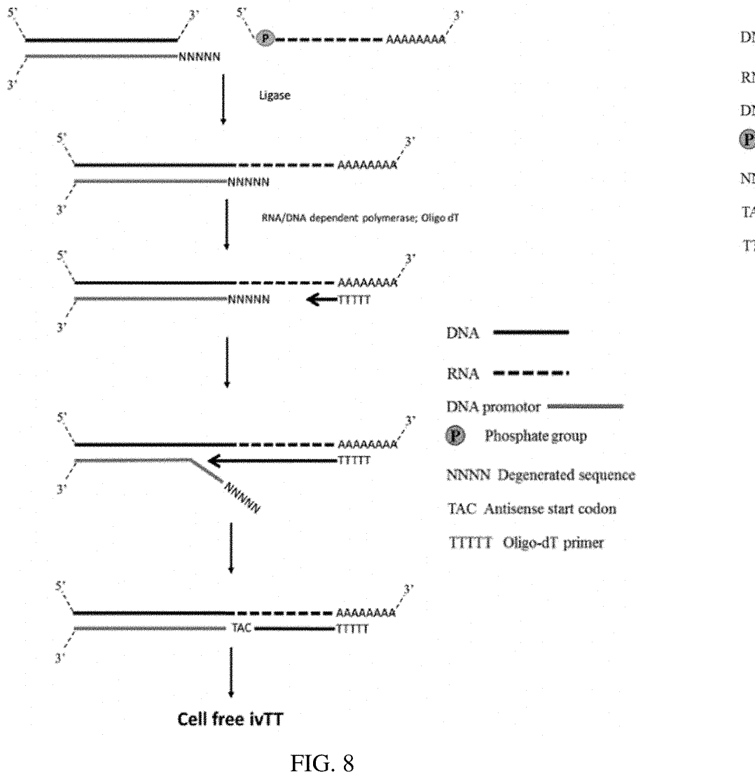

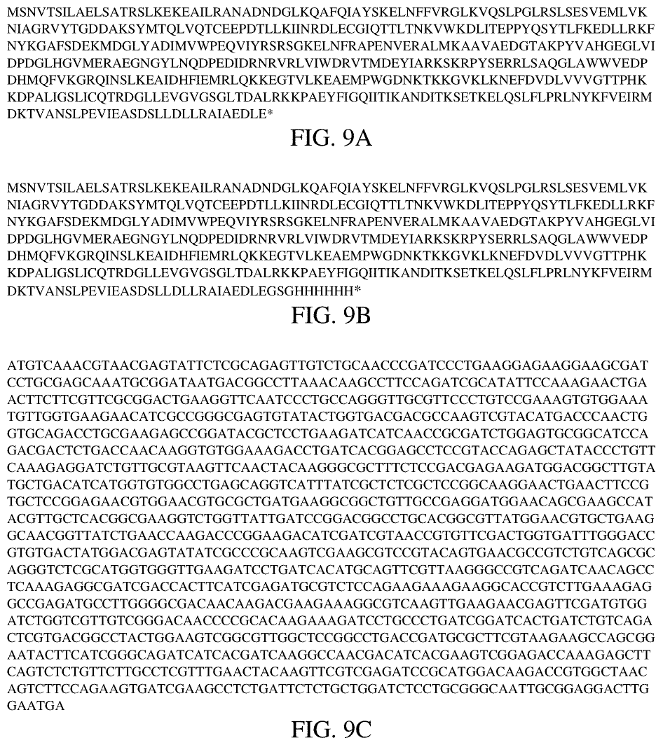

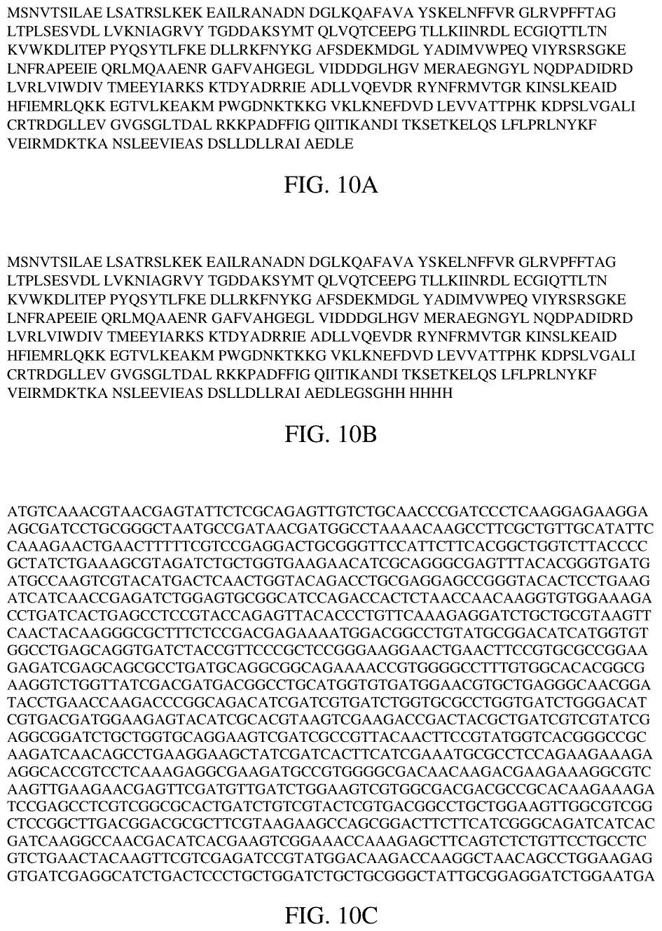

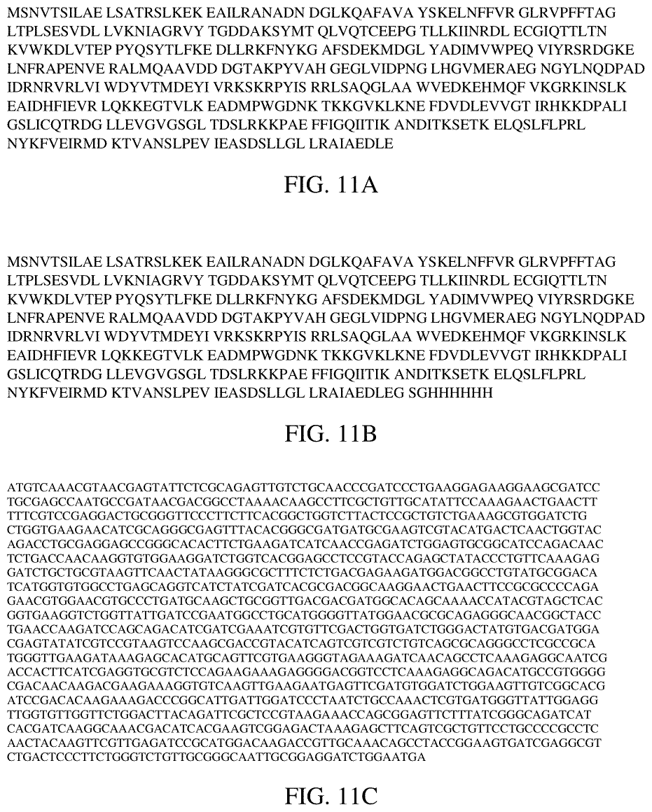

In a further embodiment the second deoxyribonucleic acid molecule may be a semi-degenerate ligation template for use in 5′end adapter ligation of RNA comprises a 3′ region that hybridizes with the first 3′-hydroxyl-deoxyribonucleic molecule, and a degenerate 5′ region comprising a random mix of nucleotides, such that each template may hybridize with a discrete RNA in the population of RNAs from different alleles. One skilled in the art will appreciate that the number of nucleotides comprising the degenerate region determines the number of possible template combinations, and hence, the number of RNAs that may be hybridized. For illustration purposes of 5′ RNA adapter ligation see FIGS. 4 to 8 where the template is semi-degenerated.

3′ Adapter Ligation of RNA

In an alternative embodiment “complementary second deoxyribonucleic acid molecule” or “complementary ligation template” is to be understood as a deoxyribonucleic acid (DNA) molecule that is used to improve efficiency of the ligation reaction. Ligation of the first 5′-phosphoryl-deoxyribonucleic acid molecule at a 3′-end of the 3′hydroxyl-ribonucleic acid molecule is performed, as described above, in the presence of this second deoxyribonucleic acid molecule also called ligation template. This second deoxyribonucleic acid molecule comprises two distinct regions: a 3′ region that is complementary to and hybridizes with the ribonucleic acid molecule and a 5′ region that is complementary to and hybridizes with the first deoxyribonucleic acid molecule

In a further embodiment the second deoxyribonucleic acid molecule may be a semi-degenerate ligation template for use in 3′end adapter ligation of RNA comprises a 5′ region that hybridizes with the first 5′-phosphoryl-deoxyribonucleic molecule, and a degenerate 3′ region comprising a random mix of nucleotides, such that each template may hybridize with a discrete RNA in the population of RNAs from different alleles. One skilled in the art will appreciate that the number of nucleotides comprising the degenerate region determines the number of possible template combinations, and hence, the number of RNAs that may be hybridized. For illustration purposes see 5′ RNA adapter ligation depicted FIGS. 4 to 8 where the template is semi-degenerated.

In certain embodiments the first 3′-hydroxyl-deoxyribonucleic acid molecule or the 5′phosphoryl-deoxyribonucleic acid molecule and the second deoxyribonucleic acid molecule are pre-hybridized in order to form a duplex described herein as an adapter molecule before adding a sample comprising ribonucleic acid molecules, for illustration of 5′ RNA adapter ligation see FIGS. 4 to 8.

The term “hybridize” is to be understood in this context a selection of hybridizing condition known in the art that are sufficient for specific annealing of complementary or approximately complementary bases on the second deoxyribonucleic acid to selectively bind and juxtaposition the two single-stranded regions of the 5′first deoxyribonucleic acid and the 3′ribonucleic acid to be joined.

The ATP-dependent DNA ligases were identified by the present inventors by mining publicly available UniProt KB database (UniProtKB/Swiss-Prot UniProt release 2015_06) comprising metagenomic nucleotide sequence data in a search for candidate ligases. Surprisingly, they discovered a sequence encoding the ATP-dependent DNA ligase according to the invention which was originally isolated from Cronobacter sakazakii bacteriophage CR9, with NCBI accession no. and Locus identity no. YP_009015226.1, having the above described unique ligase activity. The complete genome sequence of Cronobacter phage CR9 has the NCBI accession no. JQ691611. The amino acid sequence of YP_009015226.1 is recognized as a putative ligase by conceptual translation of the genome sequence, cf. the information about the sequence in the NCBI nucleotide and protein database for YP_009015226.1. According to the database the sequence has not been verified. Thus, present inventors are the first to clone and develop an optimized expression system for the ATP-dependent ligase according to the present invention.

The country of origin of the aforementioned Cronobacter phage CR9 is unclear. According to the information in the NCBI database for entry JQ691611, the Genome Sequence of Cronobacter sakazakii Bacteriophage CR9 was submitted by Department of Food and Animal Biotechnology, Seoul National University, 1 Gwanak-ro, Gwanak-gu, Seoul 151-921, South Korea.

The ability to ligate a nucleic acid molecule to the 5′end of a RNA molecule is desirable in many molecular biology techniques such as classic cloning and cloning using Gibson approach (Gibson, D. G. et al., Enzymatic assembly of DNA molecules up to several hundred kilobases, Nature Methods, 2009, vol. 6, page 343-345), adapter ligation during library preparations (e.g. illumine, Head, S. R. et al., Library construction for next-generation sequencing: overviews and challenges, Biotechniques, 2014, vo. 56, no. 2, p. 1-31), DNA synthesis, sequencing by ligation (e.g. SOLiD) (Voelkerding, K. V. et al. Next-Generation Sequencing: from basic research to diagnostics, Clinical Chemistry, 2009, vol 55, no. 4, p. 641-658), Ligase Chain reaction (J; Czajka, J; Luo, J; Barany, F; Batt, C A (February 1994). “Ligase chain reaction (LCR)—overview and applications”. PCR Methods and Applications. 3 (4): S51-64), SNP detection (Etter, P. D. et al., SNP discovery and genotyping for evolutionary genetics using RAD sequencing, Methods Mol. Biol., 2011, vol. 772, p. 157-178) and 5′-end labeling of RNA.

Commonly, 5′-adapter ligation of RNA is performed by ligating a 5′-RNA molecule or by ligating a hybrid DNA-RNA molecule to the 5′end of an RNA molecule using for example T4 RNA ligase. However, it is less favorable to work with RNA compared to DNA as RNA is more prone to degradation. Thus, it may in some context be desirable to ligate a DNA molecule to the 5′end of a RNA molecule.

Furthermore, the ligase according to the present invention has in addition to the above described unique substrate specificity also the ability to ligate with high efficiency a DNA molecule to the 3′end of a RNA molecule in the presence of a complementary DNA template that spans the ligation junction. Thus, making it possible for one enzyme to ligate DNA adapter molecules both at the 5′end and the 3′end of a RNA molecule.

The adapter ligation at the 3′ end and the 5′ end of the RNA molecule may be performed in a single step or in separate steps.

The ligase according to the present invention may therefore be used in adapter ligation processes requiring “doubly-ligated” RNA fragments (i.e. RNA fragments containing adapters ligated both at the 5′ and 3′ends). A process which is commonly used in library construction for next-generation sequencing (NGS) required for example by the widely used Illumina sequencing platform for example, see Steven R. Head et al., Library construction for next-generation sequencing: Overviews and challenges, Biotechniques, Published online 2014 Feb. 1, 2014; 56(2):61, doi: 10.2144/000114133.

RNA fragments ready for adapter ligation at the 3′ end and the 5′ end should preferably comprise a 5′ phosphoryl end-group and a 3′ hydroxyl-end group for efficient ligation to take place, for illustration of adapter ligation at the 3′ end and the 5′ end of the RNA molecule according to the present invention see figure . . . .

Long RNA molecules such as mRNA's and long non-coding RNA's may need to be fragmented. Processes for fragmentation of RNA's is known to a person skilled in the art, see for example NEBNext® RNase III RNA Fragmentation Module and NEBNext® Magnesium RNA Fragmentation Module. The NEBNext RNase III

The RNA Fragmentation Module uses a ribo-endonuclease that cleaves long double stranded RNAs into RNA fragments with 5′ phosphate and 3′ hydroxyl termini which may be used directly in 5′ and 3′end ligation reactions. The NEBNext® Magnesium RNA Fragmentation module however uses a divalent metal ion (Mg+) and heat to fragment RNA; creating RNA fragments with 5′ hydroxyl and 3′phosphate termini. In this latter case the termini of the RNA fragments must be modified in order to obtain fragments comprising a 5′ phosphate and 3′ hydroxyl termini.

It has been found that an ATP-dependent DNA ligase obtained from bacteriophage, more specifically from Cronobacter phage and as described herein surprisingly and with high efficiency ligates a DNA molecule having a single stranded region to a 5′end of RNA in the presence of a complementary DNA molecule that spans the ligation junction in the presence of ATP and a divalent cation.

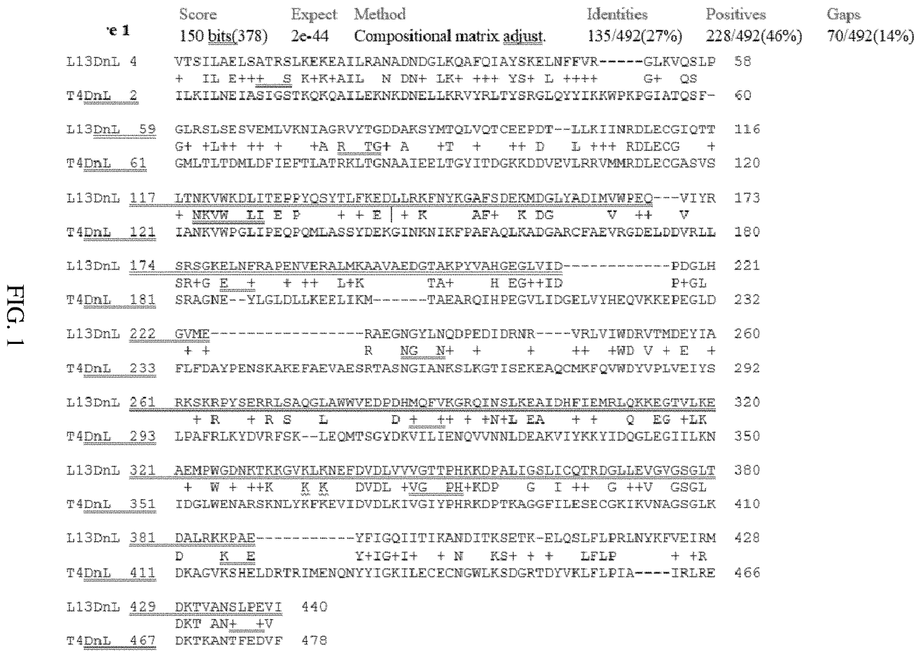

The ATP-dependent DNA ligases according to the present invention have low sequence identity with other known ligases and have less than 30% sequence identity to the well-known T4 DNA ligase enzyme, see FIG. 1.

As described above, one property of the enclosed DNA ligases is the ability to ligate with high efficiency a 3′-hydroxyl-deoxyribonucleic acid molecule to the 5′end of a 5′phosphoryl-ribonucleic acid molecule in the presence of a complementary deoxyribonucleic acid molecule that spans the ligation junction.

It is well described in literature that ligases are able to ligate a subset of different substrates, see for example Bullard, D. R., & Bowater, R. P., Direct comparison of nick-joining activity of the nucleic acid ligases from bacteriophage T4. The Biochemical Journal, 2006, 398(1), 135-144). The DNA ligases according to the invention are also able to ligate with high efficiency a subset of substrates, see FIG. 3b (AZ L13), where it is clear that AZ L13 is able to ligate with high efficacy substrate 1, 6, 7 and 8 and with a lower efficiency substrate 3 and 5.

The term high efficiency relates to the ability of the enzyme to ligate a single-stranded break in the double stranded nucleic acid molecule under standard temperature and buffer condition known to a person skilled in the art to achieve at least 50%, at least 55%, at least 60%, at least 65% at least 70%, at least 75%, at least 80%, at least 85%, at least 90%, at least 95% or at least 100% ligation of the polynucleotides within less than 6 hours, such as within less 1 hour, such as with in less than 45 minutes or preferably within 30 minutes or within 15 minutes. Examples of standard buffer and temperature conditions is described in table 2a and table 2b and example 2.

The ligation efficiency or ligation rate or conversion rate according to the present invention may be calculated using different methods known to a skilled person. For example, the ligation efficiency can be calculated by detecting the ligation products as shown in FIG. 3b, where 70% efficiency means that ((ligated product)/(ligated product+unligated product))*100=70. 70% of ligated product+non-ligated is present as ligated product. This can be detected experimentally by measuring the intensity of the different bands on a gel as shown in FIG. 3b and calculating efficiency as described. The intensity of the bands on the gel representing the ligation product and the unligated product may be measured using imaging technology for example an Alpha Imager (AlphaImager HP system).

The DNA ligase of the present invention has an improved ligation efficiency compared to T4 DNA ligase, T3DnL,SplintR or DNA ligase from Vaccinia virus in its ability to ligate a DNA molecule to the 5′end of a RNA molecule in the presence of a complementary DNA template that spans the ligation junction Wherein the improved ligation efficiency is at least 2-to 100-fold compared to the ligation efficiency of T4 DNA ligase, T3DnL,SplintR or DNA ligase from Vaccinia virus. Such that the increased ligation efficiency of the DNA ligase of the present invention compared to T4 DNA ligase, T3DnL,SplintR or DNA ligase from Vaccinia virus is as at least greater than 2-fold, at least greater than 5-fold at least greater than 10-fold, at least greater than 12-fold, at least greater than 15-fold at least greater than 20-fold or at least greater than 100-fold.

Conversion rate as a measure for enzyme activity according to the present invention may be expressed as pmol substrate ligated per pmol enzyme per minute.

The conversion rate and % ligation of the substrate is measured using a substrate comprising a single-stranded break in a double-stranded nucleic acid molecule wherein the double-stranded nucleic acid molecule comprises a 3′-hydroxyl-deoxyribonucleic acid molecule and the 5′-phosphoryl ribonucleic acid in complex with a complementary deoxyribonucleic acid molecule that spans the ligation junction (S8 substrate according to FIG. 3b).

According to the invention the conversion rate on the above described substrate (S8) is at least 0.02, at least 0.03, at least 0.04, at least 0.05 or at least 0.1 wherein the conversion rate is determined as pmol substrate per pmol enzyme per minute at 25° C. to 30° C. in a ligation buffer comprising ATP, Mg²⁺ and from 0.2 pmol to 15 pmol of a DNA ligase according to the present invention.

According to the invention the conversion rate on the above described substrate (S8) is at least 0.02 wherein the conversion rate is determined as pmol substrate per pmol enzyme per minute at 25° C. to 30° C. in a ligation buffer comprising ATP, Mg²⁺ and from 0.2 pmol to 10 pmol of a DNA ligase according to the present invention.

According to the invention the conversion rate on the above described substrate (S8) is from 0.02 to 0.1 wherein the conversion rate is determined as pmol substrate per pmol enzyme per minute at 25° C. to 30° C. in a ligation buffer comprising ATP, Mg²⁺ and from 0.2 pmol to 15 pmol of a DNA ligase according to the present invention.

According to the invention the conversion rate on the above described substrate (S8) is from 0.02 to 0.1 wherein the conversion rate is determined as pmol substrate per pmol enzyme per minute at 25° C. to 30° C. in a ligation buffer comprising ATP, Mg²⁺ and from 0.2 pmol to 10 pmol of a DNA ligase according to the present invention.

According to the invention the conversion rate on the above described substrate (S8) is at least 0.15, at least 0.2, at least 0.3, at least 0.4, at least 0.5, at least 0.8, at least 1.0, at least 1.5 wherein the conversion rate is determined as pmol substrate per pmol enzyme per minute at 25° C. to 30° C. in a ligation buffer comprising ATP, Mn²⁺ and 0.2 pmol to 15 pmol of a DNA ligase according to the present invention.

According to the invention the conversion rate on the above described substrate (S8) is at least 0.15 wherein the conversion rate is determined as pmol substrate per pmol enzyme per minute at 25° C. to 30° C. in a ligation buffer comprising ATP, Mn²⁺ and 0.2 pmol to 10 pmol of a DNA ligase according to the present invention.

According to the invention the conversion rate on the above described substrate (S8) is from 0.15 to 2.0 wherein the conversion rate is determined as pmol substrate per pmol enzyme per minute at 25° C. to 30° C. in a ligation buffer comprising ATP, Mn²⁺ and 0.2 pmol to 15 pmol of a DNA ligase according to the present invention.

According to the invention the conversion rate on the above described substrate (S8) is from 0.15 to 2.0 wherein the conversion rate is determined as pmol substrate per pmol enzyme per minute at 25° C. to 30° C. in a ligation buffer comprising ATP, Mn²⁺ and 0.2 pmol to 10 pmol of a DNA ligase according to the present invention.

The ATP dependent DNA ligases according to the present invention is able to ligate at least 25% of the above described substrate (S8) in a ligation buffer comprising ATP, Mg2⁺ and 0.2 pmol to 15 pmol of a DNA ligase according to the present invention within 15 minutes at 25° C.

The ATP dependent DNA ligases according to the present invention is able to ligate at least 25% of the above described substrate (S8) in a ligation buffer comprising ATP, Mg2⁺ and 0.2 pmol to 10 pmol of a DNA ligase according to the present invention within 15 minutes at 25° C.

The ATP dependent DNA ligases according to the present invention is able to ligate at least 50%, preferably at least 60% of the above described substrate (S8) in a ligation buffer comprising ATP, Mn²⁺ and 0.2 pmol to 15 pmol of a DNA ligase according to the present invention within 15 minutes at 25° C.

The ATP dependent DNA ligases according to the present invention is able to ligate at least 50%, preferably at least 60% of the above described substrate (S8) in a ligation buffer comprising ATP, Mn²⁺ and 0.2 pmol to 10 pmol of a DNA ligase according to the present invention within 15 minutes at 25° C.

Thus, according to one aspect of the present invention, there is provided an isolated ATP-dependent DNA ligase or an enzymatically active fragment thereof, wherein said ATP-dependent DNA ligase comprises the amino acid sequence of SEQ ID No. 1 or comprises an amino acid sequence having at least 70% amino acid sequence identity to SEQ ID No. 1 and wherein the DNA ligase is able to ligate a 3′-hydroxyl-deoxyribonucleic acid molecule at a 5′-end of a 5′phosphoryl-ribonucleic acid molecule in the presence of a complementary deoxyribonucleic acid molecule that spans a ligation junction.

The expression “an enzymatically fragment thereof” of the DNA ligase is to be understood to mean a DNA ligase wherein the catalytic activity of the ligase having an amino acid sequence as depicted in SEQ ID No. 1 is maintained in the truncated form. Example 2 provide a suitable assay to measure ligase activity.

In one embodiment the DNA ligase or an enzymatically fragment thereof according to the invention comprises an amino acid sequence which is at least 75%, 80%, 85%, 90%, 92%, 94%, 95%, 98% or 99% or preferably at least 85% or 94% identical to SEQ ID No. 1.

In some embodiments the ATP dependent DNA ligase that comprises an amino acid sequence which is at least 70% identical to SEQ ID No. 1 may be selected from sequence with:

NCBI accession number ARB11687.1,

NCBI accession number YP_007392649.1,

NCBI accession number KAB3178420.1,

NCBI accession number YP_006383262.1,

NCBI accession number YP_009042486.1,

NCBI accession number ATS93644.1,

NCBI accession number QEG12338.1,

NCBI accession number AXN57775.1 or

NCBI accession number WP_133670648.1.

In one embodiment the DNA ligase or an enzymatically fragment thereof according to the invention comprises an amino acid sequence which is at least 75% identical to SEQ ID No. 1.

In another embodiment according to the present invention the ATP dependent DNA ligase that comprises an amino acid sequence which is at least 70% identical to SEQ ID No. 1 (L13) is selected from any of the DNA ligases in Table 1:

TABLE 1

| | | | | % identity |
|---|---|---|---|---|
| | | Homologue | SEQ. | to SEQ |
| Name | NCBI Acc. No. | DNA ligases | ID. No. | ID No. 1 |
| L13Rel1 | YP_009042486.1 | DNA ligase | 7 | 84% |
| | | [Cronobacter | | |
| | | phage CR8] | | |
| L13Rel2 | YP_007392649.1 | DNA ligase | 10 | 92% |
| | | [Pectobacterium | | |
| | | phage phiTE | | |
| L13Rel4 | AXN57775.1 | DNA ligase | 16 | 76% |
| | | [Acinetobacter | | |
| | | phage ABPH49] | | |

Enclosed are also enzymatically fragments thereof of any of the above sequences of table 1.

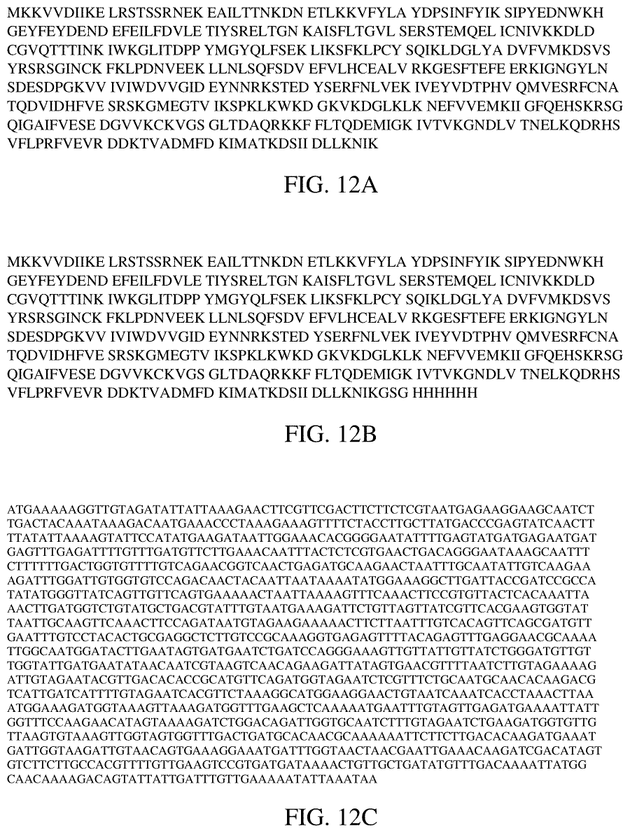

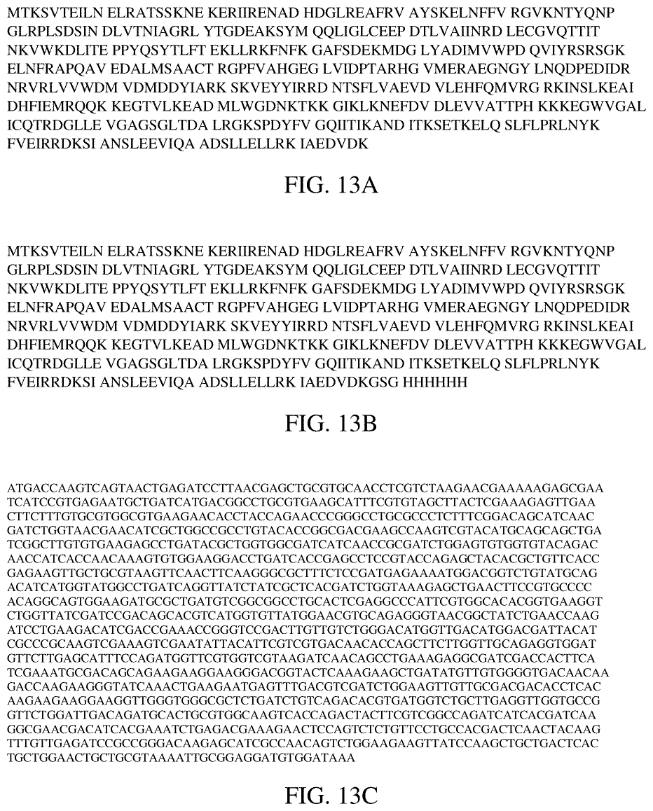

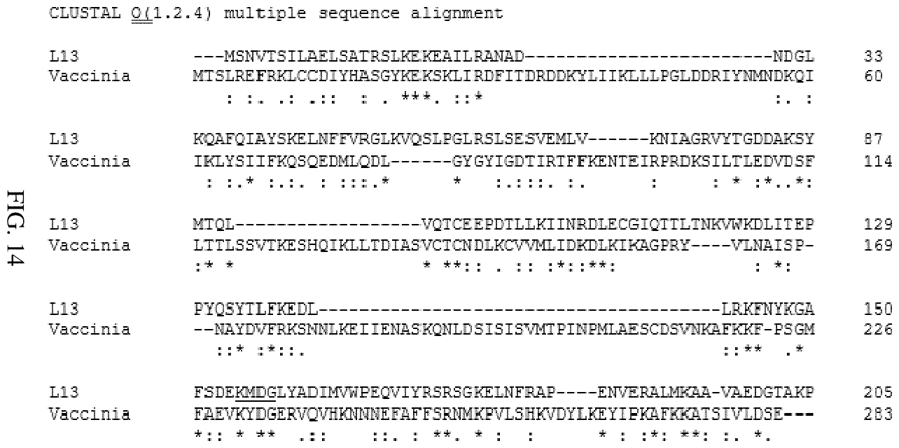

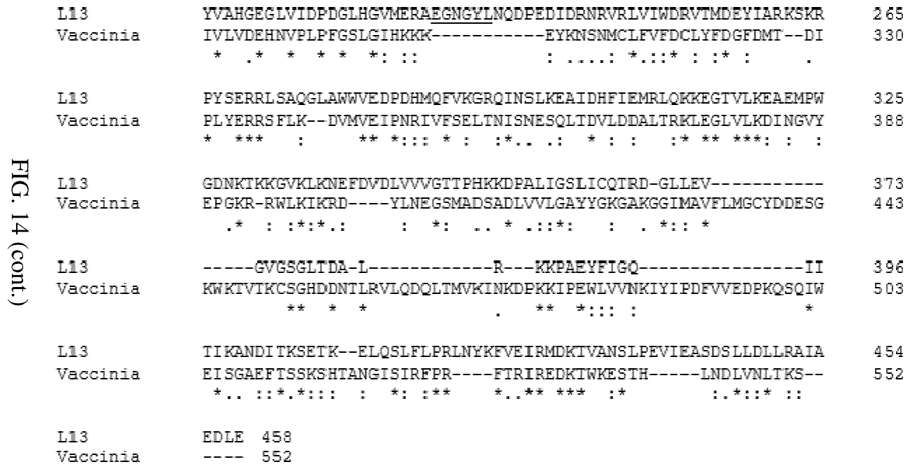

L13Rel3 with NCBI accession number YP_009846950.1, is a putative DNA ligase from Aeromonas phage 4 comprising an amino acid sequence which is only 39% identical to SEQ ID No. 1 (L13). The amino acid sequence and cDNA sequence is shown in FIGS. 12a to 12c and in SEQ ID No: 13 to 15. The L13Rel3 does not belong to the L13-cluster of DNA-dependent ligases according to the invention and the ligase as reduced enzyme activity on a substrate comprising a single-stranded break in a double-stranded nucleic acid molecule wherein the double-stranded nucleic acid molecule comprises a 3′-hydroxyl-deoxyribonucleic acid molecule and the 5′-phosphoryl ribonucleic acid in complex with a complementary deoxyribonucleic acid molecule that spans the ligation junction (S8 substrate according to FIG. 3b) compared to the L13 ATP-dependent DNA ligases according to the invention.

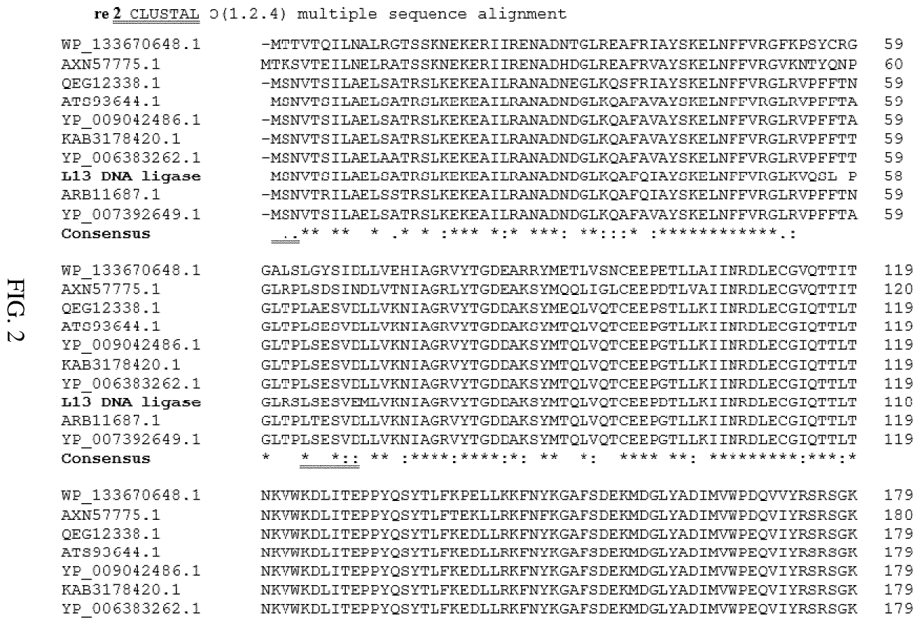

A multiple sequence alignment of SEQ ID No. 1 compared with the sequences above is shown in FIG. 2. It is apparent from FIG. 2 that the sequences share several conserved domains including the KXaaDG motif shared by all ligases investigated so far, wherein the Lysine residue is involved in forming the adenylated enzyme intermediate which then binds the nucleic acid (Tomkinson, et al., Bioessays, 19(10):893-901 (1997), Shuman, et al., Virology, 211(1):73-83 (1995), and Luo, et al., Nucleic Acids Res, 24(15):3079-3085 (1996)).

Variants of SEQ ID No. 1 include amino acid sequences in which one or more amino acids of said amino acid in SEQ ID No. 1 have undergone conservative substitutions. Preferably such substitutions are silent substitutions in that the modified form of the DNA ligase of the invention have the same enzymatic activity as the unmodified form.

The DNA ligase of the invention may be provided in a modified form, e.g. a fusion protein with an amino acid tag useful in processes for isolation, solubilization and/or purification or identification of the DNA ligase. Such amino acid tags includes, but are not limited to poly histidine (His) tags. Example of poly-Histidine tagged DNA ligase of the invention is recited in SEQ ID No. 2. Other poly-Histidine tagged DNA ligases of the invention are recited in SEQ ID No. 8, SEQ ID No. 11 and SEQ ID No. 17.

Furthermore, the present invention also provides nucleic acid molecules encoding the DNA ligase of the invention and enzymatically fragments thereof. A nucleic acid sequence corresponding to the amino acid sequence of SEQ ID No. 1 is disclosed in SEQ ID No. 3.

Further nucleic acid molecules corresponding to amino acid sequences of SEQ ID No. 7, SEQ ID No. 10 and SEQ ID No. 16 are disclosed in SEQ ID No. 9, SEQ ID No. 12 and SEQ ID No. 18.

The nucleic acid of the invention may comprise the above nucleic acid molecule or variant nucleic acid molecules. Variant nucleic acid molecules include molecules wherein a structurally different nucleotide can perform the same function or yield due to the fact that the genetic code is degenerated. Degeneracy of the genetic code means that the nucleic acid molecule of SEQ ID No. 3, SEQ ID No. 9, SEQ ID No. 12 and SEQ ID No. 18 is only one of many nucleic acid molecules encoding the amino acid molecules as provided by SEQ ID No. 1, SEQ ID No. 7, SEQ ID No. 10, and SEQ ID No. 16 without affecting the enzymatic activity of the resulting ligases. Then nucleic acid molecules disclosed in SEQ ID No. 3, SEQ ID No. 9, SEQ ID No. 12 and SEQ ID No. 18 may be codon optimized in order to optimize expression in E. coli host cell.

Disclosed are also nucleic acid molecules comprising or consisting the nucleic acid of SEQ ID No. 3, SEQ ID No. 9, SEQ ID No. 12 and SEQ ID No. 18. These nucleic acid molecules may be nucleic acid vectors, e.g. plasmids, recombinant expression vectors, viral vectors, cosmids, lambda phage vectors or bacterial artificial chromosome vectors. Preferred vectors are vectors such as plasmids for use in cloning and/or expression of the ligase in bacterial cells.

Furthermore, in other embodiment of the invention nucleic acid molecules encoding polypeptides comprising the DNA ligase and His-tags is provided. A nucleic acid molecule encoding the His-tag can be added to the nucleic acid sequences of the present invention without affecting the activity of the resulting DNA ligase.

Also, nucleic acid molecules encoding signal peptide providing for secretion of the DNA ligase enzyme from a host cell may also be linked to the nucleic acid sequences of the present invention.

As used herein, both in respect of proteins and nucleic acid molecules or fragments thereof, when referring to “sequence identity”, a sequence having at least x % identity to a second sequence means that x % represents the number of amino acids or nucleotides in the first sequence which are identical to their matched amino acids or nucleotides of the second sequence when both sequences are optimally aligned via a global alignment, relative to the total length of the second amino acid or nucleotide sequence. Both sequences are optimally aligned when x is maximum. The alignment and the determination of the percentage of identity may be carried out manually or automatically.

The skilled person will acknowledge that alignment for purposes of determining percentage sequence identity can be achieved in various ways, for instance, using publicly available computer software such as Clustal W (Thomson et al., 1994, Nucleic Acid Res., 22, pp 4673-4680) https://www.ebi.ac.uk/Tools/msa/clustalw2/NCBI BLAST (from National Center for Biotechnology Information (NCBI), USA) with default parameters.

Preparation of the DNA Ligase of the Present Invention

The DNA ligase of the present invention and the enzymatically fragments thereof or nucleic acid molecules encoding the ligase may be isolated from a natural source such as a bacteriophage, for example a Cronobacter phage, a Pectobacterium phage, or an Acinetobacter phage.

Alternatively, the enzyme may be produced recombinantly in a host cell and isolated and purified therefrom. Wherein the host cell is not, or not from an organism which naturally express the gene encoding the DNA ligase of the present invention, i.e. the host cell is a heterologous host cell such as a yeast cell, an insect cell, a human cell line or a bacterial cell, preferably E. coli.

Nucleic acid sequences encoding the DNA ligase or enzymatically fragments thereof according to the present invention may be amplified using PCR from genomic DNA, isolated as a cDNA or may be ordered by a commercial supplier such as GENEWIZ, GeneArt from Thermo Fisher Scientific or Genscript.

As described above the nucleic acid sequence encoding the DNA ligase or enzymatically fragments thereof may be codon optimized for increased protein production in a heterologous host cell. A variety of software programs for help with codon-optimization are well known in the art. CodonW is an example of an open source software program that may be used. Preferably the GeneOptimizer algorithm described by Raab, D., Graf, M., Notka, F., Schodl, T., & Wagner, R. (2010). The GeneOptimizer Algorithm: Using a sliding window approach to cope with the vast sequence space in multiparameter DNA sequence optimization. Systems and Synthetic Biology, 4(3), 215-225, is used for generating a codon optimized DNA sequence for expression of the DNA ligase of the present invention in a E. coli host cell.

There are various available molecular techniques for expression of proteins from DNA sequences by heterologous expression in various host cell systems using well known recombinant gene expression systems. For example, the nucleic acid molecule encoding the ATP-dependent DNA ligases according to the present invention or encoding an enzymatically fragment thereof may be inserted in a suitable expression vector comprising necessary transcriptional and translational elements for expression that are appropriate for the chosen host cell. Example of commonly used expression vectors are plasmids or viruses.

To ensure a reliable transcription of the gene of interest. The expression vector may comprise a strong promoter, bacteriophage T5 and T7 are examples of strong promoters for expression in E. coli. The promoter may be regulated by comprising chemical switches. Example of inducible promoters for use in E. coli is the commonly used lac promoter induced by Isoropyl-beta-D-thiogalactoside (IPTG) (Hansen L H, Knudsen S, Sorensen S J, “The effect of the lacY gene on the induction of IPTG inducible promoters, studied in Escherichia coli and Pseudomonas fluorescens”, Curr. Microbiol. 1998, 36 (6): 341-7) or the XylS/Pm expression cassette comprising a promoter that is inducible with toluic acid (Gawin, A. et al., The XylS/Pm regulator/promoter system and its use in fundamental studies of bacterial gene expression, recombinant protein production and metabolic engineering, Microb. Biotechnol., 2017, Vol. 10, No. 4, p 702-718). The XylS/Pm regulator/promoter system originating from the Pseudomonas putida is widely used for regulated low- and high-level recombinant expression of genes and gene clusters in E. coli and other bacteria.

A further aspect of the invention is a method of expression of the ATP-dependent DNA ligase according to the invention or an enzymatically fragment thereof as described above in a suitable heterologous cell. The host cell may be a bacterium or a yeast cell. Preferably the expression of the enzyme is in a bacterial host cell, more preferably E. coli, BL21 (DE3) cells.

Transformation of the above described expression vector comprising the DNA ligase of the present invention may be performed by methods well known to a skilled person, e.g. by using chemically competent cells.

As described above, the DNA ligase of the present invention may be synthesized using recombinant DNA technology. Alternatively, the DNA ligase may be produced using a cell-free expression system or chemical synthesis of the DNA ligase.

A ligase enzyme comprising a signaling peptide for secretion into cell culture media may be isolation and purified from the host cell culture media using any technique known in the art and well described in literature. Examples of such techniques or any combination may include precipitation, ultrafiltration, different chromatographic techniques e.g. size-exclusion chromatography, immobilized metal affinity column chromatography and/or immunoadsorption chromatography.

A DNA ligase enzyme of the invention produced intracellularly may be isolated and purified also using techniques well known to a skilled person. Examples of methods for preparation of cell lysates from E. coli cells are homogenization, sonication or enzymatic lysis using lysozyme. After the DNA ligase is released from the lysed cells the enzyme may be subject to any method of purification for example size-exclusion chromatography, immobilized metal affinity column chromatography and/or immunoadsorption chromatography.

As mentioned above the DNA ligase of the present invention may comprise a c-terminal His-tag to ease isolation, purification and/or identification of the enzyme. Polyhistidine tagged DNA ligases of the invention is depicted in SEQ ID No. 2, SEQ ID No. 8, SEQ ID No. 11 and SEQ ID No. 17.

Thus, according to another aspect of the present invention, there is provided method for isolation and purification of the DNA ligase or the enzymatically active fragment thereof according to the present invention, comprising the steps of:

a) culturing the host cell under conditions suitable for expressing the ATP-dependent DNA ligase according to the present invention;

b) isolating the DNA ligase from the host cell or from the culture medium or supernatant.

The purified ATP-dependent DNA ligase enzyme or an enzymatically fragment thereof according to the present invention may finally be stored in a buffer comprising for example 10 mM Tris-HCl pH 7.5 (25° C.), 0.3 M KCl, 5 mM MgCl₂, 0.2 mg/ml BSA and 50% glycerol. Alternative buffers suitable for storing the ligase is known to a skilled person.

Compositions and Kits Comprising the DNA Ligase of the Present Invention

Disclosed are also compositions and kits comprising the ATP-dependent DNA ligase according to the first aspect and one or more additional necessary reagents to carry out the ligation step, for example a ligation buffer. A suitable ligation buffer and reaction conditions for carrying out the ligation is described in table 2a or 2b, below.

Typically, the isolated ATP-dependent DNA ligase of the invention will be placed in an aqueous buffer similar a composition comprising the isolated ATP-dependent DNA ligase of the invention will also comprise an aqueous buffer. The aqueous buffer according to the invention comprising a standard buffer such as Tris, MES Bis-Tris, Phosphate or HEPES buffers with a pH from about 7 to about 8.5, preferably pH about 7.5. The aqueous buffer may preferably further comprise BSA and glycerol for stabilizing the enzyme.

The DNA ligases of the present invention are ATP-dependent DNA ligases that utilizes ATP as a cofactor in order to carry out the ligation step, see paragraph “background” for an overview of the enzymatic reaction. Furthermore, the ATP-dependency of the reaction indicates that the reaction requires multiple divalent cations such as Mg²⁺ or Mn²⁺ ions for catalysis and that an essential divalent cation, preferably Mg²⁺, more preferably Mn²⁺ is required.

Thus, in one embodiment the ligation buffer comprises ATP. Such that wherein the concentration of ATP is from about 0.01 mM to about 10 mM and preferably from about 0.05 mM to about 2.5 mM and more preferably about 1 mM.

In a further embodiment the ligation buffer comprises a divalent cation. Such that wherein the divalent cation is Mg²⁺ or Mn²⁺ for example in form of MgCl₂ or MnCl₂.

In a further embodiment the concentration of the divalent cation in the ligation buffer is between 1 mM to 20 mM, preferably between 5 mM and 15 mM and more preferably about 10 mM.

The ligation buffer may further comprise a reducing agent. Non-limiting examples of suitable reducing agents include dithiothreitol and [beta]-mercaptoethanol.

In a further embodiment the ligation is performed at a temperature between 20° C. to 35° C. and preferably between 25° C. to 30° C.

Also disclosed in a further aspect is a kit comprising any of the isolated ATP-dependent DNA ligases according to the invention. The kit may further comprise a ligation buffer for optimal ligation. The kit may also comprise a written description on how to perform the ligation step using the disclosed ATP-dependent DNA ligase according to the invention. Suitable conditions are described in example 2, which may be provided in the kit or together with the ligase enzyme. Table 2a and 2b provides examples of reaction mixtures for optimal ligation using the DNA ligases of the present invention.

TABLE 2a

| | Concentration/volume | |
|---|---|---|
| Tris/HCl (pH 7.4) | 55 | mM |
| MgCl₂ | 10 | mM |
| DTT | 10.5 | mM |
| KCl | 25 | mM |
| ATP | 1 | mM |
| Ligase | 70 | pmol |
| Double stranded nucleic | 45 | pmol |
| acid comprising a nick | | |
| Total volume | 5 | μl |

TABLE 2b

| | Concentration/volume |
|---|---|
| Tris/HCl (pH 7.4) | 50 mM |
| MnCl₂ | 10 mM |
| DTT | 10 mM |
| KCl | 25 mM |
| ATP | 0.1 to 1 mM |
| Ligase | 0.2 pmol to 15 pmol |
| Double stranded nucleic | from 5 to 45 pmol |
| acid comprising a nick | |
| Total volume | from 5 μl to 20 μl |

The reaction mixture in table 2a or table 2b is incubated at 30° C. for 30 minutes. Alternatively, the reaction mixture in table 2a or table 2b is incubated at 25° C. for 15 minutes. The reaction mixture of table 2a or 2b may comprise either MnCl₂ or MgCl₂.

Use of the DNA Ligase of the Invention

In another aspect of the present invention there is provided a method for ligating a single stranded break in a double stranded nucleic acid molecule wherein said method comprising contacting the double stranded nucleic acid molecule comprising a single stranded break with the isolated ATP-dependent DNA ligase or enzymatically active fragment thereof according to the first aspect under conditions which permits ligation of the 3′-hydroxyl-deoxyribonucleic acid molecule to the 5′end of the 5′-phosphoryl ribonucleic acid molecule in the double stranded nucleic acid molecule wherein 3′-hydroxyl-deoxyribonucleic acid molecule and the 5′-phosphoryl ribonucleic acid is in complex with a complementary deoxyribonucleic acid molecule that spans the ligation junction.

In one embodiment the ATP-dependent DNA ligase according to the present invention or enzymatically active fragment thereof is the L13 ligase.

In one embodiment the ATP-dependent DNA ligase according to the present invention or enzymatically active fragment thereof is the L13rel1 ligase.

In one embodiment the ATP-dependent DNA ligase according to the present invention or enzymatically active fragment thereof is the L13rel2 ligase.

In one embodiment the ATP-dependent DNA ligase according to the present invention or enzymatically active fragment thereof is the L13rel4 ligase.

A single stranded-break according to the invention comprises both a nick and a gap.

5′-phosphoryl-ribonucleic acid molecule is an RNA molecule comprising a phosphate group on the 5′end. In one embodiment the ribonucleic acid molecule may be a ribonucleic acid (RNA) molecule selected from a group comprising messenger RNA (mRNA), microRNA (miRNA), short interfering RNA (siRNA), repeat associated siRNA (rasiRNA).

The source of a small RNA-containing sample that is suitable for use in this invention can and will vary depending upon the application. The sample comprising a mature small RNA may be derived from animals, plants, fungi, protists, viruses, bacteria, or archaea.

The sample derived from any of the aforementioned sources may range from a preparation of essentially pure RNA molecules to a crude extract of a cell. In one embodiment, the sample may be an isolated preparation of small RNA molecules. In another embodiment, the sample may be an isolated preparation of total RNA extracted from a cell. In yet another embodiment, the sample may be a cytosolic cellular extract comprising nucleic acids, proteins, lipids, and carbohydrates. In still another embodiment, the sample may be an intact cell. In yet another embodiment, the sample comprising the small RNA may be an in vitro transcription reaction or a chemical synthesis reaction. Total RNA or small RNA may be isolated and purified from cells, cellular extracts, or in vitro reactions using commercially available kits or techniques well known in the art, for reference, see Ausubel et al. (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y.).

The amount of small RNA in the sample added to a ligation reaction can and will vary depending upon the source of the RNA-containing sample. In general, any amount of RNA may be used.

Messenger RNA (mRNA) usually has a five-prime cap (5′ cap), which is a specially altered nucleotide on the 5′ end of some primary transcripts such as precursor messenger RNA. This process, known as mRNA capping, is highly regulated and vital in the creation of stable and mature messenger RNA able to undergo translation during protein synthesis. Mitochondrial mRNA and chloroplastic mRNA are not capped. In order for ligation to take place the mRNA may first need to be de-Capped and then phosphorylated on the 5′end. For illustration see FIG. 7. Suitable de-Capping enzymes are known to a person skilled in the art. FIG. 6 illustrates a situation where mRNA's comprising a 5′cap is not ligated.

Examples of suitable conditions for performing the method is described in table 2 above and in example 2.

In one embodiment according to the above method further comprises incubating the ATP dependent DNA ligase together with the double stranded nucleic acid molecule comprising a single stranded break to achieve at least 50%, at least 55%, at least 60%, at least 65%, at least 70%, at least 75%, at least 80%, at least 85%, at least 90%, at least 95% or at least 100% ligation of the polynucleotides within less than 6 hours, such as within less 1 hour, such as with in less than 45 minutes or preferably within 30 minutes such as within 15 minutes.

In an alternative embodiment of the above method the 3′-hydroxyl-deoxyribonucleic acid molecule is immobilized on a bead or further comprises a capturing label preferably biotin or a derivatized nucleotide such as a dye, preferably a fluorescent dye wherein the bead, the capturing label or the derivatized nucleotide is linked to the 5′end of said deoxyribonucleic acid molecule.

Assays for detecting the ligation product is known to a person skilled in the art. Examples of traditional methods of detecting ligation products include denaturing gel electrophoresis, sequence amplification, and melting curve analysis.

### EXAMPLES

The invention will now be described by way of non-limiting examples with reference to the above figures and biological sequences of table 7.

The ATP-dependent DNA ligase (L13) of the present invention was discovered by a sequence-based metagenomic approach mining public databases for candidate ligases. As mentioned above the DNA ligase of the present invention was originally isolated from Cronobacter phage CR9 and its DNA and protein sequence can be obtained from NCBI Reference Sequence: YP_009015226.1. Further preferred ATP-dependent DNA ligases according to the present invention are homologue DNA ligases of DNA ligase L13 with an amino acid sequence identity of at least 70% to the L13 ligase and originally isolated from: Cronobacter phage CR8 and its DNA and protein sequence can be obtained from NCBI Reference Sequence: YP_009042486.1 (L13rel1); originally isolated from Pectobacterium phage phiTE and its DNA and protein sequence can be obtained from NCBI Reference Sequence: YP_007392649.1 (L13rel2); originally isolated from Acinetobacter phage ABPH49 and its DNA and protein sequence can be obtained from NCBI Reference Sequence: AXN57775.1 (L13rel4).

### Example 1 Cloning, Expression and Purification

Selected sequences were codon-optimized for expression in E. coli, cloned into the expression vector pVB-1A0B1 (Vectron Biosolutions) with a C-terminal His-tag and transformed into E. coli BL21 (DE3).

Cloning

The coding sequence of L13 or any of L13rel1, L13rel2, L13rel3 or L13rel4 were codon optimized for expression in E. coli using the GeneOptimizer algorithm (Raab, D., Graf, M., Notka, F., Schödl, T., & Wagner, R. (2010). The GeneOptimizer Algorithm: Using a sliding window approach to cope with the vast sequence space in multiparameter DNA sequence optimization. Systems and Synthetic Biology, 4(3), 215-225. http://doi.org/10.1007/s11693-010-9062-3) and ordered using the GeneArt gene synthesis service from Thermo Fisher Scientific. Flanking the gene coding sequence additional sequence information was added coding for a C-terminal GSG linker and His-tag followed by a stop-codon, N-terminal PciI and NdeI restriction sites and a C-terminal XhoI restriction site for downstream cloning approaches. The NdeI and XhoI restriction sites were used for cloning into the expression vector pVB-1A0B1 (Vectron Biosolutions, Trondheim, Norway). The pVB vector family consists of a proprietary E. coli expression vector backbone based on the RK2 plasmid with the XylS/Pm expression cassette comprising a promoter that is inducible with toluic acid. The XylS/Pm regulator/promoter system originating from the Pseudomonas putida TOL plasmid pWW0 is widely used for regulated low- and high-level recombinant expression of genes and gene clusters in Escherichia coli (E. coli) and other bacteria and is described in Gawin, A. et al., The XylS/Pm regulator/promoter system and its use in fundamental studies of bacterial gene expression, recombinant protein production and metabolic engineering, Microb. Biotechnol., 2017, Vol. 10, No. 4, p 702-718.

Expression and Purification of the DNA Ligases

A codon optimized nucleic acid molecule selected from SEQ ID No. 3, SEQ ID No. 9, SEQ ID No. 12, SEQ ID No. 15 or SEQ ID No. 18 was cloned into the expression vector pVB-1A0B1 from Vectron Biosolutions and transformed into BL21 (DE3) cells. The cells were grown in 2.5 L Ultra Yield (Thomson) flasks with Terrific Broth (TB) medium; 1% overnight precultures were transferred to 1 L TB medium containing 100 μg/ml Ampicillin and incubated at 37° C., 220 rpm, until the OD600 reached 5-6. The temperature was decreased to 15° C. and when the temperature was ≤20° C. the cells were induced with 2 mM Toluic acid. The cells were incubated overnight (ON), harvested by centrifugation and frozen at −20° C.

Frozen cell pellets were added lysis buffer (50 mM Tris-HCl (pH 8.5 at 25° C.), 10 mM imidazole, 0.5 M NaCl, 5 mM MgCl2, 0.5% Tween 20, 5% glycerol, 1 mg/ml lysozyme and 400 U/ml HL-SAN) to an OD600 of 120 and incubated over night at 15° C. with 90 rpm. The lysates were centrifuged for 20 minutes at 20 000 g and filtered before purification.

The first purification step was performed using a HiScale 26/20 column packed with 33.4 ml Ni-Sepharose 6 FF. After application of the lysate the column was washed with IMAC wash buffer (50 mM Tris-HCl (pH 7.5 at 25° C.), 20 mM Imidazole, 5 mM MgCl2 and 0.5 M NaCl) before elution with increasing concentrations of imidazole. In the second purification step a HiScale 26/20 column packed with 34.5 ml Q-Sepharose FF resin was used. After application of diluted eluate from the first purification step the column was washed with Q wash buffer (20 mM Tris-HCl pH 7.5 (25° C.) and 50 mM KCl) and the His-tagged L13 enzyme was eluted from the column using a Q-elution buffer (20 mM Tris-HCl pH 7.5 (25° C.), 10 mM MgCl2 and 0.2 M NaCl). The purified L13 ligase enzyme was finally stored in 10 mM Tris-HCl pH 7.5 (25° C.), 0.3 M KCl, 5 mM MgCl2, 0.2 mg/ml BSA and 50% glycerol.

### Example 2A Ligase Activity Assay for Measuring Substrate Specificity

Experimental Setup

Ligase activity on different substrates may be assayed according to the procedure of Bullard and Bowater (Bullard, D. R., & Bowater, R. P. (2006). Direct comparison of nick-joining activity of the nucleic acid ligases from bacteriophage T4. The Biochemical Journal, 398(1), 135-144). An in vitro assay analyzing the ligation activity were performed, using a double-stranded substrate and was based on a modified setup described in Bullard and Bowater 2006. Oligonucleotides were purchased from Metabion (Germany). The double-stranded substrates were generated by annealing one 8-mer nucleic acid oligo and one 12-mer nucleic acid oligo to a complementary 20-mer nucleic acid template oligo using a 30 solution of each oligo. The annealing step was performed in 100 μl TE buffer (10 mM Tris/HCl, pH 8, and 0.5 mM EDTA) and the buffer comprising the three oligoes was heated to 95° C. for 5 minutes and cooled down at room temperature for 16 hours. The 5′nucleotide residue of the 8-mer oligo is labeled with a fluorescein dye molecule 6-FAM (IUPAC name 3′,6′-dihydroxy spiro[isobenzofuran-1(3H),9′-[9H]xanthen]-3-one; CAS number 2321-07-5) and the 12-mer oligo comprises a 5′-monophosphorylated nucleic acid residue. Each double-stranded substrate was represented by either ribonucleic acid oligonucleotides only or deoxyribonucleic acid oligonucleotides only or a mixture of ribonucleic acid oligonucleotides and deoxyribonucleic acid oligonucleotides, leading to in total of eight different compositions of double-stranded substrates as depicted in table 3 bellow and in FIGS. 3a and 3b.

8-mer oligo*)
(SEQ ID No. 4)
5′-GGCCAGTG-3′
12-mer oligo*)
(SEQ ID No. 5)
5′-AATTCGAGCTCG-3′
20-mer oligo*)
(SEQ ID No. 6)
5′-CGAGCTCGAATTCACTGGCC-3′

The T's are replaced by U's in the RNA oligos.

The nicked double-stranded 20 base pair (bp) substrates were used in an end-point in vitro assay measuring the ligation activity of each enzyme as depicted in FIG. 3a. The buffer used in all ligation reactions comprises 55 mM Tris/HCl (pH 7.4), 10 mM MgCl₂, 10.5 mM DTT 25 mM KCl and 1 mM ATP. The end-point ligation reactions were performed as followed: 70 pmol of a ligase, 45 pmol of a double-stranded oligo nucleic acid substrate, as described above, in a total volume of 5 μl. The reaction mixture was incubated at 30° C. for 30 min and then stopped using 5 μl of a formamide stop solution (95% Formamide, 10 mM EDTA, brome-phenol-blue). At the end of the experiments, samples were heated to 95° C. for 5 minutes and analyzed under denaturating conditions on a 20% polyacrylamide-urea gel 1×TBE (89 mM Tris, 89 mM boric acid and 2 mM EDTA). The reaction products, see FIG. 3b, were visualized on the gel using a Bio-Rad Pharos system. Quantification was performed using ImageLab (BioRad).

The ligation activity of the L13 DNA ligase compared to other commercially available ligases were analyzed as illustrated in FIG. 1b. Surprisingly, the L13 DNA ligase was the only ligase being able to ligate a single-stranded break in substrate 8 (S8) with high efficiency. Substrate S8 was a poor substrate for the commercially available ligase enzymes tested.

### Example 2B Ligase Activity Assay on an Alternative S8 Like Substrate

The ligation activity of the L13 DNA ligase on an alternative S8 substrate was tested. The nicked double-stranded 43 base pair (bp) substrate, comprising the sequences with SEQ ID No 19, 20 and 21, were used in an end-point in vitro assay measuring the ligation activity of L13. The buffer used in all ligation reactions comprises 50 mM Tris/HCl (pH 7.5), 10 mM MgCl₂, 10 mM DTT, <46 mM KCl and 0.1 mM ATP. The end-point ligation reactions were performed as followed: different amounts of L13 ligase, 9 pmol of a double-stranded oligo nucleic acid substrate, as described above, in a total volume of 10 μl. The reaction mixture was incubated at 25° C. for 15 min and then stopped using 5 μl of a formamide stop solution (95% Formamide, 10 mM EDTA, brome-phenol-blue). At the end of the experiments, samples were heated to 95° C. for 5 minutes and analyzed under denaturating conditions on a 20% polyacrylamide-urea gel 1×TBE (89 mM Tris, 89 mM boric acid and 2 mM EDTA). The reaction products, were visualized on the gel using a Bio-Rad Pharos system. Quantification was performed using ImageLab (BioRad).

The results from the experiments are depicted in table 3 below clearly demonstrates that the L13 ligase is also able to ligate with high efficiency a nick in a double stranded DNA-RNA hybrid comprising different nucleic acid sequences compared to example 2A.

Alternative S8-substrate
20-mer 5′-FAM-labelled DNA oligo
(SEQ ID No. 19)
5′-GGCCAGTGAATTCGAGCTCG-3′
23-mer RNA oligo 5′-P
(SEQ ID No. 20)
5′-P-ACUGAUAGAAGUUCCCGAAACAG-3′
43-mer DNA oligo
(SEQ ID No. 21)
5′-CTGTTTCGGGAACTTCTATCAGTCGAGCTCGAATTCACTGGCC-3′

TABLE 3

| | | | pmol | Conversion rate |
|---|---|---|---|---|
| L13 | KCl | % | substrate | (pmol substrate/(pmol |
| (pmol) | (mM) | ligation | ligated | enzyme per min)) |
| 63 | 45.8 | 62.2 | 5.6 | Outside linear range |
| 21 | 31.9 | 50.0 | 4.5 | Outside linear range |
| 7 | 27.3 | 34.0 | 3.1 | 0.029 |
| 2.3 | 25.8 | 11.0 | 0.99 | 0.028 |

### Example 3 Ligase Activity of L13, L13Rel1, L13rel2, L13rel3 and L13rel4

In this example the ligase activity of the different ligases of the present invention were tested. The results are shown in table 4 and table 5 and demonstrates that L13, L13rel1, L13rel2 and L13rel4 enzymes have similar ligation efficiency on the S8 substrate, i.e. ligating a DNA molecule to the 5′-end of a RNA molecule in the presence of a DNA molecule that spans the ligation junction.

TABLE 4

| | Condition 1 | | Condition 2 | | Conversion rate |
|---|---|---|---|---|---|
| | | pmol | | pmol | (pmol substrate |
| | % | substrate | % | substrate | per pmol enzyme |
| Ligase | ligation | ligated | ligation | ligated | per minute) |
| Negative | 0.71 | 0.06 | 1.24 | 0.22 | 0.002 |
| control | | | | | |
| L13 | 81.0 | 7.3 | 39.4 | 7.09 | 0.07 |
| L13rel1 | 79.2 | 7.1 | 34.3 | 6.17 | 0.06 |
| L13rel4 | 75.5 | 6.8 | 22.9 | 4.12 | 0.04 |

Assay condition 1: 50 mM Tris/HCl pH 7.5, 10 mM MgCl2, 10 mM DTT, 25 mM KCl, 1 mM ATP, 11.4 pmol ligase, 9 pmol S8 substrate, total volume 20 μl. Incubation 30 minutes at 30° C.

Assay condition 2: 50 mM Tris/HCl pH 7.5, 10 mM MgCl₂, 10 mM DTT, 25 mM KCl, 1 mM ATP, 7 pmol ligase, 18 pmol S8 substrate, total volume 20 μl. Incubation 15 minutes at 25° C.

TABLE 5

| | | pmol |
|---|---|---|
| | % | substrate |
| | ligation | ligated |
| Negative control | 1.8 | 0.32 |
| L13 | 73.0 | 13.2 |
| L13rel1 | 65.2 | 11.7 |
| L13rel2 | 71.2 | 12.8 |
| L13rel3 | 24.5 | 4.4 |
| L13rel4 | 69.8 | 12.6 |

Assay conditions: 50 mM Tris/HCl pH 7.5, 10 mM MnCl₂, 10 mM DTT, 25 mM KCl, 1 mM ATP, 11.4 pmol ligase, 18 pmol S8 substrate, total volume 20 μl. Incubation 15 minutes at 25° C.

### Example 4 Ligase Activity of L13 DNA Ligase Compared to Vaccinia DNA Ligase

The efficacy of the L13 DNA ligase of the present invention was compared to the DNA ligase from Vaccinia virus of prior art (the protein sequence of the Vaccinia DNA ligase is depicted in the NCBI database with accession number YP_233058.1. Table 6 depicts the results from the experiments. Enzyme activity was tested using 18 pmol S8 substrate according to example 2a; a ligation buffer comprising either MnCl₂ or MgCl₂ and a reaction volume of 20 μl. The ligation was run at 25° C. for 15 min and % ligated substrate and conversion rate was calculated.

TABLE 6

| | | | | | Conversion rate |
|---|---|---|---|---|---|
| | | | pmol | | (pmol substrate |
| | | % ligated | ligated | pmol | per pmol enzyme |
| Enzyme | Buffer | substrate | substrate | ligase | per minute) |
| L13 | MnCl₂ | 52.4 | 9.4 | 0.4 | 1.6 |
| Vaccinia | MnCl₂ | 35.8 | 6.4 | 4 | 0.1 |
| L13 | MgCl₂ | 25.8 | 4.6 | 4 | 0.08 |
| Vaccinia | MgCl₂ | 8.4 | 1.5 | 15.6 | 0.006 |

The data presented in table 6 clearly demonstrate that L13 is more efficient ligase compare to Vaccinia DNA ligase in ligating a DNA molecule to the 5′-end of a RNA molecule in the presence of a DNA molecule that spans the ligation junction (for substrate S8, see example 2).

TABLE 7

| SEQ | |
|---|---|
| ID No. | Sequence information |
| 1 | L13 DNA ligase protein sequence |
| 2 | L13 DNA ligase protein sequence including His-tag |
| 3 | cDNA sequence encoding L13 DNA ligase of SEQ ID No. 1 |
| 4 | 8-mer oligo |
| 5 | 12-mer oligo |
| 6 | 20-mer oligo |
| 7 | L13rel1 DNA ligase protein sequence |
| 8 | L13rel1 DNA ligase protein sequence including His-tag |
| 9 | cDNA sequence encoding L13rel1 DNA ligase |
| 10 | L13rel2 DNA ligase protein sequence including |
| 11 | L13rel2 DNA ligase protein sequence including His-tag |
| 12 | cDNA sequence encoding L13rel2 DNA ligase |
| 13 | L13rel3 including ligase protein sequence |
| 14 | L13rel3 including ligase protein sequence including His-tag |
| 15 | cDNA sequence encoding L13rel3 DNA ligase |
| 16 | L13rel4 DNA ligase protein sequence |
| 17 | L13rel4 DNA ligase protein sequence including His-tag |
| 18 | cDNA sequence encoding L13rel4 DNA ligase |
| 19 | 20-mer 5′-FAM labelled DNA oligo |
| 29 | 23-mer RNA oligo 5′-P |
| 21 | 43-mer DNA oligo |

1. An isolated ATP-dependent DNA ligase or an enzymatically active fragment thereof, wherein the DNA ligase comprises an amino acid sequence of SEQ ID No. 1 or comprises an amino acid sequence having at least 70% amino acid sequence identity to SEQ ID No. 1 and wherein the DNA ligase is able to ligate a 3′-hydroxyl-deoxyribonucleic acid molecule to a 5′-end of a 5′phosphoryl-ribonucleic acid molecule in the presence of a complementary deoxyribonucleic acid molecule that spans the ligation junction.

2. The isolated ATP-dependent DNA ligase or an enzymatically active fragment thereof according to claim 1, wherein the DNA ligase comprises an amino acid sequence having at least 75% amino acid sequence identity to SEQ ID No. 1.

3. The isolated ATP-dependent DNA ligase or an enzymatically active fragment according to claim 1, wherein the DNA ligase is able to ligate a 3′-hydroxyl-deoxyribonucleic acid molecule to a 5′-end of a 5′phosphoryl-ribonucleic acid molecule in the presence of a complementary deoxyribonucleic acid molecule that spans the ligation junction, wherein the DNA ligase has an amino acid sequence selected from the group consisting of:
a. SEQ ID No. 1 or an amino acid sequence having at least 80% identity thereto,
b. SEQ ID No. 7 or an amino acid sequence having at least 80% identity thereto,
c. SEQ ID No. 10 or an amino acid sequence having at least 80% identity thereto, and
d. SEQ ID No. 16 or an amino acid sequence having at least 80% identity thereto.

4. The isolated ATP-dependent DNA ligase or an enzymatically active fragment thereof according to claim 1, wherein said DNA ligase has an amino acid sequence selected from the group consisting of SEQ ID No. 1, SEQ ID No. 2, SEQ ID No. 7, SEQ ID No. 8, SEQ ID No. 10, SEQ ID No. 11, SEQ ID No. 16 and SEQ ID No. 17.

5. A recombinant nucleic acid molecule encoding the isolated ATP-dependent DNA ligase or an enzymatically active fragment thereof according to claim 1 or encoding a protein comprising said isolated ATP-dependent DNA ligase or an enzymatically active fragment thereof.

6. The recombinant nucleic acid molecule of claim 5, wherein said nucleic acid molecule comprises a sequence selected from the group consisting of SEQ ID No. 3, SEQ ID No. 9, SEQ ID No. 12, SEQ ID No. 18, a codon-optimized sequence of SEQ ID No. 3, a codon-optimized sequence of SEQ ID No. 9, a codon-optimized sequence of SEQ ID No. 12, a codon-optimized sequence of SEQ ID No. 18, a degenerated version of SEQ ID NO:3, a degenerated version of SEQ ID NO:9, a degenerated version of SEQ ID NO:12, and a degenerated version of SEQ ID NO:18.

7. (canceled)

8. A vector comprising a nucleic acid molecule encoding the isolated ATP-dependent DNA ligase or an enzymatically active fragment thereof according to claim 1 or a nucleic acid molecule encoding a protein comprising said isolated ATP-dependent DNA ligase or an enzymatically active fragment thereof, wherein the vector is a recombinant expression vector, a cloning vector, a plasmid, a viral vector, a cosmid, a lambda phage or a bacterial artificial chromosome.

9. A host cell comprising the vector according to claim 8, wherein the cell is a yeast cell, insect cell, a human cell line or bacterial cell.

10. A composition comprising the isolated ATP-dependent DNA ligase or an enzymatically fragment thereof according to claim 1.

11. The composition according to claim 10, wherein the composition further comprises a buffer, wherein said buffer comprises ATP and a divalent cation and wherein the divalent cation is Mn²⁺ or Mg²⁺.

12. The composition according to claim 10, wherein the composition is for a ligation of nucleic acid molecules wherein the ligation is an RNA 5′-end adapter ligation, a ligation for capturing RNA molecules of known or unknown sequences, or a ligation of an DNA element that serves as a template in a cDNA molecule synthesis comprising promoter elements and/or translation enhancer elements to 5′ends of RNA molecules for the purpose of in vitro transcription.

13. The composition according to claim 10, wherein the composition further comprises at least one first 3′-hydroxyl-deoxyribonucleic acid molecule, at least one 5′phosphoryl-ribonucleic molecule and at least one second complementary deoxyribonucleic acid molecule wherein the DNA ligase is able to ligate the at least one first 3′-hydroxyl-deoxyribonucleic acid molecule to the 5′end of the at least one 5′phosphoryl-ribonucleic molecule in the presence of the at least one complementary deoxyribonucleic acid molecule that spans the ligation junction.

14. A kit for ligating a deoxyribonucleic acid molecule to a terminus of a ribonucleic acid molecule comprising:
a. a first container comprising the isolated ATP-dependent DNA ligase or an enzymatically fragment thereof according to claim 1 or a composition thereof;
b. a second container comprising a ligation buffer wherein the buffer comprises ATP and a divalent cation;
c. optionally a third container comprising at least one first 3′-hydroxyl-deoxyribonucleic acid molecule to be ligated to a 5′end of a 5′-phosphoryl-ribonucleic acid molecule, and at least one second deoxyribonucleic molecule wherein the at least one second deoxyribonucleic acid molecule comprises a 3′region and a 5′region wherein the 3′region is complementary to the first deoxyribonucleic acid molecule and the 5′ region is either a sequence that is complementary to a ribonucleic acid molecule comprising a known sequence or the 5′region is a sequence that is degenerated in order to bind ribonucleic acid molecules having an unknown sequence or comprising different sequences and wherein the first deoxyribonucleic molecule and the second deoxyribonucleic molecule may be in the form of a prehybridized complex; and
d. optionally instructions for using the kit.

15. The kit according to claim 14, wherein the kit further comprises a fourth container comprising at least one first 5′-phosphoryl-deoxyribonucleic acid molecule to be ligated to a 3′end of a 3′-hydroxyl-ribonucleic acid molecule, and at least one second deoxyribonucleic molecule wherein the at least one second deoxyribonucleic acid molecule comprises a 3′region and a 5′region wherein the 5′region is complementary to the first 5′-phosphoryl-deoxyribonucleic acid molecule and wherein the 3′ region is a sequence that is complementary to a ribonucleic acid molecule when the sequence is a known sequence or the 3′region is a sequence that is degenerated in order to bind ribonucleic acid molecules when the sequence is an unknown sequence or comprises different sequences and wherein the first 5′-phosphoryl-deoxyribonucleic acid and the second deoxyribonucleic molecule may be in the form of a prehybridized complex.

16. A method for ligating a single-stranded break in a double-stranded nucleic acid molecule wherein said method comprising contacting the double-stranded nucleic acid molecule comprising a single stranded break with the isolated ATP-dependent DNA ligase or enzymatically active fragment thereof according to claim 1 or a composition thereof under conditions which permits ligation of a 3′-hydroxyl-deoxyribonucleic acid molecule to a 5′end of a 5′-phosphoryl ribonucleic acid molecule in the double stranded nucleic acid molecule wherein the 3′-hydroxyl-deoxyribonucleic acid molecule and the 5′-phosphoryl ribonucleic acid is in complex with a complementary deoxyribonucleic acid molecule that spans the ligation junction.

17. (canceled)

18. A method for ligating a single-stranded break in a double-stranded nucleic acid molecule according to claim 16, wherein the 5′phosphoryl-deoxyribonucleic acid molecule and the ribonucleic acid molecule are in complex with a complementary deoxyribonucleic acid molecule that spans the ligation junction.

19. The method according to claim 16 wherein the composition comprising the double-stranded nucleic acid molecule comprising a single stranded break further comprises ATP and a divalent cation, wherein the divalent cation is Mn²⁺ or Mg²⁺.

20. (canceled)

21. A method for ligating a deoxyribonucleic acid molecule to a 5′end and a 3′end of ribonucleic acid molecules, the method comprising:
a. providing a sample comprising a population of ribonucleic acid molecules wherein one or more of the ribonucleic acid molecules comprises a 5′phosphoryl-end group and a 3′-hydroxyl-end group;
b. ligating at least one first 3′-hydroxyl-deoxyribonucleic acid molecule to the 5′end of the ribonucleic acid molecules in the presences of at least one second deoxyribonucleic wherein the at least one second deoxyribonucleic acid molecule comprises a 3′region and a 5′ region wherein the 3′region is complementary to the first deoxyribonucleic acid molecule and wherein the 5′ region is a sequence that is complementary to a ribonucleic acid molecule comprising a known sequence or the 5′region is a sequence that is degenerated in order to bind ribonucleic acid molecules having an unknown sequence or comprising different sequences, and wherein the first deoxyribonucleic molecule and the second deoxyribonucleic molecule may be in the form of a prehybridized complex; and
c. ligating at least one other 5′phosphoryl-deoxyribonucleic acid molecule to the 3′-end of the ribonucleic acid molecules in step b. in the presences of at least one additional second deoxyribonucleic comprises a 3′region and a 5′ region wherein the 5′region is complementary to the 5′phosphoryl-deoxyribonucleic acid molecule and wherein the 3′ region is either a sequence that is complementary to the ribonucleic acid molecule comprising a known sequence in step b or the 3′region is a sequence that is degenerated in order to bind the ribonucleic acid molecules having an unknown sequence or comprising different sequences, and wherein the 5′phosphoryl-deoxyribonucleic acid and the additional second deoxyribonucleic molecule may be in the form of a prehybridized complex; and
wherein the ligation reactions in steps b and c are catalyzed by the ATP-dependent ligase according to claim 1 or a composition thereof and wherein the ligation reactions in steps b and c are performed simultaneously or sequentially, and
wherein the sample further comprises ATP and a divalent cation, wherein the cation is Mn²⁺ or Mg²⁺.

22. (canceled)

23. A recombinant nucleic acid molecule encoding the isolated ATP-dependent DNA ligase or an enzymatically active fragment thereof according to claim 4 or encoding a protein comprising said isolated ATP-dependent DNA ligase or an enzymatically active fragment thereof.


SEQUENCE LISTING (21 sequences)

SEQ ID NO: 1 - protein, 458 aa, L13 wild type amino acid sequence
MSNVTSILAELSATRSLKEKEAILRANADNDGLKQAFQIAYSKELNFFVRGLKVQSLPGLRSLSESVEMLVKNIAGRVYTGDDAKSYMTQLVQTCEEPDTLLKIINRDLECGIQTTLTNKVWKDLITEPPYQSYTLFKEDLLRKFNYKGAFSDEKMDGLYADIMVWPEQVIYRSRSGKELNFRAPENVERALMKAAVAEDGTAKPYVAHGEGLVIDPDGLHGVMERAEGNGYLNQDPEDIDRNRVRLVIWDRVTMDEYIARKSKRPYSERRLSAQGLAWWVEDPDHMQFVKGRQINSLKEAIDHFIEMRLQKKEGTVLKEAEMPWGDNKTKKGVKLKNEFDVDLVVVGTTPHKKDPALIGSLICQTRDGLLEVGVGSGLTDALRKKPAEYFIGQIITIKANDITKSETKELQSLFLPRLNYKFVEIRMDKTVANSLPEVIEASDSLLDLLRAIAEDLE

SEQ ID NO: 2 - protein, 467 aa, L13 amino acid sequence with His-tag
MSNVTSILAELSATRSLKEKEAILRANADNDGLKQAFQIAYSKELNFFVRGLKVQSLPGLRSLSESVEMLVKNIAGRVYTGDDAKSYMTQLVQTCEEPDTLLKIINRDLECGIQTTLTNKVWKDLITEPPYQSYTLFKEDLLRKFNYKGAFSDEKMDGLYADIMVWPEQVIYRSRSGKELNFRAPENVERALMKAAVAEDGTAKPYVAHGEGLVIDPDGLHGVMERAEGNGYLNQDPEDIDRNRVRLVIWDRVTMDEYIARKSKRPYSERRLSAQGLAWWVEDPDHMQFVKGRQINSLKEAIDHFIEMRLQKKEGTVLKEAEMPWGDNKTKKGVKLKNEFDVDLVVVGTTPHKKDPALIGSLICQTRDGLLEVGVGSGLTDALRKKPAEYFIGQIITIKANDITKSETKELQSLFLPRLNYKFVEIRMDKTVANSLPEVIEASDSLLDLLRAIAEDLEGSGHHHHHH

SEQ ID NO: 3 - DNA, 1377 bp, L13 cDNA sequence
atgtcaaacgtaacgagtattctcgcagagttgtctgcaacccgatccctgaaggagaaggaagcgatcctgcgagcaaatgcggataatgacggccttaaacaagccttccagatcgcatattccaaagaactgaacttcttcgttcgcggactgaaggttcaatccctgccagggttgcgttccctgtccgaaagtgtggaaatgttggtgaagaacatcgccgggcgagtgtatactggtgacgacgccaagtcgtacatgacccaactggtgcagacctgcgaagagccggatacgctcctgaagatcatcaaccgcgatctggagtgcggcatccagacgactctgaccaacaaggtgtggaaagacctgatcacggagcctccgtaccagagctataccctgttcaaagaggatctgttgcgtaagttcaactacaagggcgctttctccgacgagaagatggacggcttgtatgctgacatcatggtgtggcctgagcaggtcatttatcgctctcgctccggcaaggaactgaacttccgtgctccggagaacgtggaacgtgcgctgatgaaggcggctgttgccgaggatggaacagcgaagccatacgttgctcacggcgaaggtctggttattgatccggacggcctgcacggcgttatggaacgtgctgaaggcaacggttatctgaaccaagacccggaagacatcgatcgtaaccgtgttcgactggtgatttgggaccgtgtgactatggacgagtatatcgcccgcaagtcgaagcgtccgtacagtgaacgccgtctgtcagcgcagggtctcgcatggtgggttgaagatcctgatcacatgcagttcgttaagggccgtcagatcaacagcctcaaagaggcgatcgaccacttcatcgagatgcgtctccagaagaaagaaggcaccgtcttgaaagaggccgagatgccttggggcgacaacaagacgaagaaaggcgtcaagttgaagaacgagttcgatgtggatctggtcgttgtcgggacaaccccgcacaagaaagatcctgccctgatcggatcactgatctgtcagactcgtgacggcctactggaagtcggcgttggctccggcctgaccgatgcgcttcgtaagaagccagcggaatacttcatcgggcagatcatcacgatcaaggccaacgacatcacgaagtcggagaccaaagagcttcagtctctgttcttgcctcgtttgaactacaagttcgtcgagatccgcatggacaagaccgtggctaacagtcttccagaagtgatcgaagcctctgattctctgctggatctcctgcgggcaattgcggaggacttggaatga

SEQ ID NO: 4 - DNA, 8 bp, 8-mer DNA oligo
ggccagtg

SEQ ID NO: 5 - RNA, 12 nt, 12-mer RNA oligo
aauucgagcucg

SEQ ID NO: 6 - DNA, 20 bp, 20-mer DNA oligo
cgagctcgaattcactggcc

SEQ ID NO: 7 - protein, 455 aa, L13Rel1
MSNVTSILAELSATRSLKEKEAILRANADNDGLKQAFAVAYSKELNFFVRGLRVPFFTAGLTPLSESVDLLVKNIAGRVYTGDDAKSYMTQLVQTCEEPGTLLKIINRDLECGIQTTLTNKVWKDLITEPPYQSYTLFKEDLLRKFNYKGAFSDEKMDGLYADIMVWPEQVIYRSRSGKELNFRAPEEIEQRLMQAAENRGAFVAHGEGLVIDDDGLHGVMERAEGNGYLNQDPADIDRDLVRLVIWDIVTMEEYIARKSKTDYADRRIEADLLVQEVDRRYNFRMVTGRKINSLKEAIDHFIEMRLQKKEGTVLKEAKMPWGDNKTKKGVKLKNEFDVDLEVVATTPHKKDPSLVGALICRTRDGLLEVGVGSGLTDALRKKPADFFIGQIITIKANDITKSETKELQSLFLPRLNYKFVEIRMDKTKANSLEEVIEASDSLLDLLRAIAEDLE

SEQ ID NO: 8 - protein, 464 aa, L13Rel1amino acid sequence with His-tag
MSNVTSILAELSATRSLKEKEAILRANADNDGLKQAFAVAYSKELNFFVRGLRVPFFTAGLTPLSESVDLLVKNIAGRVYTGDDAKSYMTQLVQTCEEPGTLLKIINRDLECGIQTTLTNKVWKDLITEPPYQSYTLFKEDLLRKFNYKGAFSDEKMDGLYADIMVWPEQVIYRSRSGKELNFRAPEEIEQRLMQAAENRGAFVAHGEGLVIDDDGLHGVMERAEGNGYLNQDPADIDRDLVRLVIWDIVTMEEYIARKSKTDYADRRIEADLLVQEVDRRYNFRMVTGRKINSLKEAIDHFIEMRLQKKEGTVLKEAKMPWGDNKTKKGVKLKNEFDVDLEVVATTPHKKDPSLVGALICRTRDGLLEVGVGSGLTDALRKKPADFFIGQIITIKANDITKSETKELQSLFLPRLNYKFVEIRMDKTKANSLEEVIEASDSLLDLLRAIAEDLEGSGHHHHHH

SEQ ID NO: 9 - DNA, 1368 bp, L13Rel1 cDNA
atgtcaaacgtaacgagtattctcgcagagttgtctgcaacccgatccctcaaggagaaggaagcgatcctgcgggctaatgccgataacgatggcctaaaacaagccttcgctgttgcatattccaaagaactgaactttttcgtccgaggactgcgggttccattcttcacggctggtcttaccccgctatctgaaagcgtagatctgctggtgaagaacatcgcagggcgagtttacacgggtgatgatgccaagtcgtacatgactcaactggtacagacctgcgaggagccgggtacactcctgaagatcatcaaccgagatctggagtgcggcatccagaccactctaaccaacaaggtgtggaaagacctgatcactgagcctccgtaccagagttacaccctgttcaaagaggatctgctgcgtaagttcaactacaagggcgctttctccgacgagaaaatggacggcctgtatgcggacatcatggtgtggcctgagcaggtgatctaccgttcccgctccgggaaggaactgaacttccgtgcgccggaagagatcgagcagcgcctgatgcaggcggcagaaaaccgtggggcctttgtggcacacggcgaaggtctggttatcgacgatgacggcctgcatggtgtgatggaacgtgctgagggcaacggatacctgaaccaagacccggcagacatcgatcgtgatctggtgcgcctggtgatctgggacatcgtgacgatggaagagtacatcgcacgtaagtcgaagaccgactacgctgatcgtcgtatcgaggcggatctgctggtgcaggaagtcgatcgccgttacaacttccgtatggtcacgggccgcaagatcaacagcctgaaggaagctatcgatcacttcatcgaaatgcgcctccagaagaaagaaggcaccgtcctcaaagaggcgaagatgccgtggggcgacaacaagacgaagaaaggcgtcaagttgaagaacgagttcgatgttgatctggaagtcgtggcgacgacgccgcacaagaaagatccgagcctcgtcggcgcactgatctgtcgtactcgtgacggcctgctggaagttggcgtcggctccggcttgacggacgcgcttcgtaagaagccagcggacttcttcatcgggcagatcatcacgatcaaggccaacgacatcacgaagtcggaaaccaaagagcttcagtctctgttcctgcctcgtctgaactacaagttcgtcgagatccgtatggacaagaccaaggctaacagcctggaagaggtgatcgaggcatctgactccctgctggatctgctgcgggctattgcggaggatctggaatga

SEQ ID NO: 10 - protein, 459 aa, L13Rel2 amino acid sequence
MSNVTSILAELSATRSLKEKEAILRANADNDGLKQAFAVAYSKELNFFVRGLRVPFFTAGLTPLSESVDLLVKNIAGRVYTGDDAKSYMTQLVQTCEEPGTLLKIINRDLECGIQTTLTNKVWKDLVTEPPYQSYTLFKEDLLRKFNYKGAFSDEKMDGLYADIMVWPEQVIYRSRDGKELNFRAPENVERALMQAAVDDDGTAKPYVAHGEGLVIDPNGLHGVMERAEGNGYLNQDPADIDRNRVRLVIWDYVTMDEYIVRKSKRPYISRRLSAQGLAAWVEDKEHMQFVKGRKINSLKEAIDHFIEVRLQKKEGTVLKEADMPWGDNKTKKGVKLKNEFDVDLEVVGTIRHKKDPALIGSLICQTRDGLLEVGVGSGLTDSLRKKPAEFFIGQIITIKANDITKSETKELQSLFLPRLNYKFVEIRMDKTVANSLPEVIEASDSLLGLLRAIAEDLE

SEQ ID NO: 11 - protein, 468 aa, L13Rel2 amino acid sequence with His-tag
MSNVTSILAELSATRSLKEKEAILRANADNDGLKQAFAVAYSKELNFFVRGLRVPFFTAGLTPLSESVDLLVKNIAGRVYTGDDAKSYMTQLVQTCEEPGTLLKIINRDLECGIQTTLTNKVWKDLVTEPPYQSYTLFKEDLLRKFNYKGAFSDEKMDGLYADIMVWPEQVIYRSRDGKELNFRAPENVERALMQAAVDDDGTAKPYVAHGEGLVIDPNGLHGVMERAEGNGYLNQDPADIDRNRVRLVIWDYVTMDEYIVRKSKRPYISRRLSAQGLAAWVEDKEHMQFVKGRKINSLKEAIDHFIEVRLQKKEGTVLKEADMPWGDNKTKKGVKLKNEFDVDLEVVGTIRHKKDPALIGSLICQTRDGLLEVGVGSGLTDSLRKKPAEFFIGQIITIKANDITKSETKELQSLFLPRLNYKFVEIRMDKTVANSLPEVIEASDSLLGLLRAIAEDLEGSGHHHHHH

SEQ ID NO: 12 - DNA, 1380 bp, L13Rel2 cDNA
atgtcaaacgtaacgagtattctcgcagagttgtctgcaacccgatccctgaaggagaaggaagcgatcctgcgagccaatgccgataacgacggcctaaaacaagccttcgctgttgcatattccaaagaactgaactttttcgtccgaggactgcgggttcccttcttcacggctggtcttactccgctgtctgaaagcgtggatctgctggtgaagaacatcgcagggcgagtttacacgggcgatgatgcgaagtcgtacatgactcaactggtacagacctgcgaggagccgggcacacttctgaagatcatcaaccgagatctggagtgcggcatccagacaactctgaccaacaaggtgtggaaggatctggtcacggagcctccgtaccagagctataccctgttcaaagaggatctgctgcgtaagttcaactataagggcgctttctctgacgagaagatggacggcctgtatgcggacatcatggtgtggcctgagcaggtcatctatcgatcacgcgacggcaaggaactgaacttccgcgccccagagaacgtggaacgtgccctgatgcaagctgcggttgacgacgatggcacagcaaaaccatacgtagctcacggtgaaggtctggttattgatccgaatggcctgcatggggttatggaacgcgcagagggcaacggctacctgaaccaagatccagcagacatcgatcgaaatcgtgttcgactggtgatctgggactatgtgacgatggacgagtatatcgtccgtaagtccaagcgaccgtacatcagtcgtcgtctgtcagcgcagggcctcgccgcatgggttgaagataaagagcacatgcagttcgtgaagggtagaaagatcaacagcctcaaagaggcaatcgaccacttcatcgaggtgcgtctccagaagaaagaggggacggtcctcaaagaggcagacatgccgtggggcgacaacaagacgaagaaaggtgtcaagttgaagaatgagttcgatgtggatctggaagttgtcggcacgatccgacacaagaaagacccggcattgattggatccctaatctgccaaactcgtgatgggttattggaggttggtgttggttctggacttacagattcgctccgtaagaaaccagcggagttctttatcgggcagatcatcacgatcaaggcaaacgacatcacgaagtcggagactaaagagcttcagtcgctgttcctgccccgcctcaactacaagttcgttgagatccgcatggacaagaccgttgcaaacagcctaccggaagtgatcgaggcgtctgactcccttctgggtctgttgcgggcaattgcggaggatctggaatga

SEQ ID NO: 13 - protein, 457 aa, L13Rel3 amino acid sequence
MKKVVDIIKELRSTSSRNEKEAILTTNKDNETLKKVFYLAYDPSINFYIKSIPYEDNWKHGEYFEYDENDEFEILFDVLETIYSRELTGNKAISFLTGVLSERSTEMQELICNIVKKDLDCGVQTTTINKIWKGLITDPPYMGYQLFSEKLIKSFKLPCYSQIKLDGLYADVFVMKDSVSYRSRSGINCKFKLPDNVEEKLLNLSQFSDVEFVLHCEALVRKGESFTEFEERKIGNGYLNSDESDPGKVVIVIWDVVGIDEYNNRKSTEDYSERFNLVEKIVEYVDTPHVQMVESRFCNATQDVIDHFVESRSKGMEGTVIKSPKLKWKDGKVKDGLKLKNEFVVEMKIIGFQEHSKRSGQIGAIFVESEDGVVKCKVGSGLTDAQRKKFFLTQDEMIGKIVTVKGNDLVTNELKQDRHSVFLPRFVEVRDDKTVADMFDKIMATKDSIIDLLKNIK

SEQ ID NO: 14 - protein, 466 aa, L13Rel3 amino acid sequence with His-tag
MKKVVDIIKELRSTSSRNEKEAILTTNKDNETLKKVFYLAYDPSINFYIKSIPYEDNWKHGEYFEYDENDEFEILFDVLETIYSRELTGNKAISFLTGVLSERSTEMQELICNIVKKDLDCGVQTTTINKIWKGLITDPPYMGYQLFSEKLIKSFKLPCYSQIKLDGLYADVFVMKDSVSYRSRSGINCKFKLPDNVEEKLLNLSQFSDVEFVLHCEALVRKGESFTEFEERKIGNGYLNSDESDPGKVVIVIWDVVGIDEYNNRKSTEDYSERFNLVEKIVEYVDTPHVQMVESRFCNATQDVIDHFVESRSKGMEGTVIKSPKLKWKDGKVKDGLKLKNEFVVEMKIIGFQEHSKRSGQIGAIFVESEDGVVKCKVGSGLTDAQRKKFFLTQDEMIGKIVTVKGNDLVTNELKQDRHSVFLPRFVEVRDDKTVADMFDKIMATKDSIIDLLKNIKGSGHHHHHH

SEQ ID NO: 15 - DNA, 1374 bp, L13Rel3 cDNA
atgaaaaaggttgtagatattattaaagaacttcgttcgacttcttctcgtaatgagaaggaagcaatcttgactacaaataaagacaatgaaaccctaaagaaagttttctaccttgcttatgacccgagtatcaacttttatattaaaagtattccatatgaagataattggaaacacggggaatattttgagtatgatgagaatgatgagtttgagattttgtttgatgttcttgaaacaatttactctcgtgaactgacagggaataaagcaatttcttttttgactggtgttttgtcagaacggtcaactgagatgcaagaactaatttgcaatattgtcaagaaagatttggattgtggtgtccagacaactacaattaataaaatatggaaaggcttgattaccgatccgccatatatgggttatcagttgttcagtgaaaaactaattaaaagtttcaaacttccgtgttactcacaaattaaacttgatggtctgtatgctgacgtatttgtaatgaaagattctgttagttatcgttcacgaagtggtattaattgcaagttcaaacttccagataatgtagaagaaaaacttcttaatttgtcacagttcagcgatgttgaatttgtcctacactgcgaggctcttgtccgcaaaggtgagagttttacagagtttgaggaacgcaaaattggcaatggatacttgaatagtgatgaatctgatccagggaaagttgttattgttatctgggatgttgttggtattgatgaatataacaatcgtaagtcaacagaagattatagtgaacgttttaatcttgtagaaaagattgtagaatacgttgacacaccgcatgttcagatggtagaatctcgtttctgcaatgcaacacaagacgtcattgatcattttgtagaatcacgttctaaaggcatggaaggaactgtaatcaaatcacctaaacttaaatggaaagatggtaaagttaaagatggtttgaagctcaaaaatgaatttgtagttgagatgaaaattattggtttccaagaacatagtaaaagatctggacagattggtgcaatctttgtagaatctgaagatggtgttgttaagtgtaaagttggtagtggtttgactgatgcacaacgcaaaaaattcttcttgacacaagatgaaatgattggtaagattgtaacagtgaaaggaaatgatttggtaactaacgaattgaaacaagatcgacatagtgtcttcttgccacgttttgttgaagtccgtgatgataaaactgttgctgatatgtttgacaaaattatggcaacaaaagacagtattattgatttgttgaaaaatattaaataa

SEQ ID NO: 16 - protein, 457 aa, L13Rel4 amino acid sequence
MTKSVTEILNELRATSSKNEKERIIRENADHDGLREAFRVAYSKELNFFVRGVKNTYQNPGLRPLSDSINDLVTNIAGRLYTGDEAKSYMQQLIGLCEEPDTLVAIINRDLECGVQTTITNKVWKDLITEPPYQSYTLFTEKLLRKFNFKGAFSDEKMDGLYADIMVWPDQVIYRSRSGKELNFRAPQAVEDALMSAACTRGPFVAHGEGLVIDPTARHGVMERAEGNGYLNQDPEDIDRNRVRLVVWDMVDMDDYIARKSKVEYYIRRDNTSFLVAEVDVLEHFQMVRGRKINSLKEAIDHFIEMRQQKKEGTVLKEADMLWGDNKTKKGIKLKNEFDVDLEVVATTPHKKKEGWVGALICQTRDGLLEVGAGSGLTDALRGKSPDYFVGQIITIKANDITKSETKELQSLFLPRLNYKFVEIRRDKSIANSLEEVIQAADSLLELLRKIAEDVDK

SEQ ID NO: 17 - protein, 466 aa, L13Rel4 amino acid sequence with His-tag
MTKSVTEILNELRATSSKNEKERIIRENADHDGLREAFRVAYSKELNFFVRGVKNTYQNPGLRPLSDSINDLVTNIAGRLYTGDEAKSYMQQLIGLCEEPDTLVAIINRDLECGVQTTITNKVWKDLITEPPYQSYTLFTEKLLRKFNFKGAFSDEKMDGLYADIMVWPDQVIYRSRSGKELNFRAPQAVEDALMSAACTRGPFVAHGEGLVIDPTARHGVMERAEGNGYLNQDPEDIDRNRVRLVVWDMVDMDDYIARKSKVEYYIRRDNTSFLVAEVDVLEHFQMVRGRKINSLKEAIDHFIEMRQQKKEGTVLKEADMLWGDNKTKKGIKLKNEFDVDLEVVATTPHKKKEGWVGALICQTRDGLLEVGAGSGLTDALRGKSPDYFVGQIITIKANDITKSETKELQSLFLPRLNYKFVEIRRDKSIANSLEEVIQAADSLLELLRKIAEDVDKGSGHHHHHH

SEQ ID NO: 18 - DNA, 1371 bp, L13Rel4 cDNA
atgaccaagtcagtaactgagatccttaacgagctgcgtgcaacctcgtctaagaacgaaaaagagcgaatcatccgtgagaatgctgatcatgacggcctgcgtgaagcatttcgtgtagcttactcgaaagagttgaacttctttgtgcgtggcgtgaagaacacctaccagaacccgggcctgcgccctctttcggacagcatcaacgatctggtaacgaacatcgctggccgcctgtacaccggcgacgaagccaagtcgtacatgcagcagctgatcggcttgtgtgaagagcctgatacgctggtggcgatcatcaaccgcgatctggagtgtggtgtacagacaaccatcaccaacaaagtgtggaaggacctgatcaccgagcctccgtaccagagctacacgctgttcaccgagaagttgctgcgtaagttcaacttcaagggcgctttctccgatgagaaaatggacggtctgtatgcagacatcatggtatggcctgatcaggttatctatcgctcacgatctggtaaagagctgaacttccgtgccccacaggcagtggaagatgcgctgatgtcggcggcctgcactcgaggcccattcgtggcacacggtgaaggtctggttatcgatccgacagcacgtcatggtgttatggaacgtgcagagggtaacggctatctgaaccaagatcctgaagacatcgaccgaaaccgggtccgacttgttgtctgggacatggttgacatggacgattacatcgcccgcaagtcgaaagtcgaatattacattcgtcgtgacaacaccagcttcttggttgcagaggtggatgttcttgagcatttccagatggttcgtggtcgtaagatcaacagcctgaaagaggcgatcgaccacttcatcgaaatgcgacagcagaagaaggaagggacggtactcaaagaagctgatatgttgtggggtgacaacaagaccaagaagggtatcaaactgaagaatgagtttgacgtcgatctggaagttgttgcgacgacacctcacaagaagaaggaaggttgggtgggcgctctgatctgtcagacacgtgatggtctgcttgaggttggtgccggttctggattgacagatgcactgcgtggcaagtcaccagactacttcgtcggccagatcatcacgatcaaggcgaacgacatcacgaaatctgagacgaaagaactccagtctctgttcctgccacgactcaactacaagtttgttgagatccgccgggacaagagcatcgccaacagtctggaagaagttatccaagctgctgactcactgctggaactgctgcgtaaaattgcggaggatgtggataaa

SEQ ID NO: 19 - DNA, 20 bp, 20-mer DNA oligo
ggccagtgaattcgagctcg

SEQ ID NO: 20 - RNA, 23 nt, 23-mer RNA oligo
acugauagaaguucccgaaacag

SEQ ID NO: 21 - DNA, 43 bp, 43-mer DNA oligo
ctgtttcgggaacttctatcagtcgagctcgaattcactggcc